SEQUENCE LISTING (28 sequences)

SEQ ID NO: 1 - DNA, 28 bp, Borago officinalis
ccaatcatcctacgaggtacttgtccag

SEQ ID NO: 2 - DNA, 28 bp, Borago officinalis
gcaaaaactcctccctgttcatgcctag

SEQ ID NO: 3 - DNA, 26 bp, Borago officinalis
acgtgcttcctaggcatgaacaggga

SEQ ID NO: 4 - DNA, 28 bp, Borago officinalis
acacggtgaactcgtctaaccttgagga

SEQ ID NO: 5 - DNA, 32 bp, Borago officinalis
gaagccatggggatctcctcacatttcacaac

SEQ ID NO: 6 - DNA, 29 bp, Borago officinalis
ttgtccaacaccggtacttatgattgcat

SEQ ID NO: 7 - DNA, 30 bp, Borago officinalis
tagcatctccaaacgttttaaatgtcaacg

SEQ ID NO: 8 - DNA, 27 bp, Borago officinalis
tcctcgtagatgattggtactgatgcg

SEQ ID NO: 9 - DNA, 24 bp, Borago officinalis
gcctggcccggttttgattgacgt

SEQ ID NO: 10 - DNA, 27 bp, Borago officinalis
tgaaatacaacgcaagtcaaactctac

SEQ ID NO: 11 - DNA, 23 bp, Borago officinalis
tctccaccactctcttcaccgtc

SEQ ID NO: 12 - DNA, 77 bp, Borago officinalis
tattggatgtcattgtcccacatcaagaacatgtgttgcctatgatcccaagtggcggaacctttgatgatgttatc

SEQ ID NO: 13 - DNA, 77 bp, Borago officinalis
tattggatgtcgttgtgccacatcaagaacatgtgctgcctatgatcccaagtggcggaacctttgacgatgttatt

SEQ ID NO: 14 - DNA, 77 bp, Borago officinalis
  mutation of nucleotide (52)..(52): G to A
tattggatgtcattgtcccacatcaagaacatgtgttgcctatgatcccaaatggcggaacctttgatgatgttatc

SEQ ID NO: 15 - DNA, 77 bp, Borago officinalis
  nucleotide mutation (52)..(52): G to A
tattggatgtcgttgtgccacatcaagaacatgtgctgcctatgatcccaaatggcggaacctttgacgatgttatt

SEQ ID NO: 16 - protein, 670 aa, Borago officinalis
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 17 - protein, 652 aa, Borago officinalis
MAAPPNPSISFKPPSPAAALPPRSAFLPRFALPITSTTQKRHRLHISNVLSDSKSTTTTTQPPLQAQPFVSRYAPDQPRKGADVLVEALEREGVTDVFAYPGGASMEIHQALTRSNTIRNVLPRHEQGGVFAAEGYARASGLPGVCIATSGPGATNLVSGLADALLDSVPMVAITGQVPRRMIGTDVFQETPIVEVTRSITKHNYLVLDVEDIPRIVREAFYLASSGRPGPVLIDVPKDIQQQLVVPKWDEPMRLPGYLSRMPKPQYDGHLEQIVRLVGEAKRPVLYVGGGCLNSDDELRRFVELTGIPVASTLMGLGAYPASSDLSLHMLGMHGTVYANYAVDKSDLLLAFGVRFDDRVTGKLEAFASRAKIVHIDIDPAEIGKNKQPHVSICGDIKVALQGLNKILEEKNSVTNLDFSNWRKELDEQKVKFPLSFKTFGEAIPPQHAIQVLDELTGGNAIISTGVGQHQMWAAQFYKYNKPRQWLTSGGLGAMGFGLPAAIGAAVARPDAVVVDIDGDGSFMMNVQELATIRVENLPVKILLLNNQHLGMVVQWEDRFYKANRAHTYLGNPSKESEIFPNMVKFAEACDIPAARVTQKADLRAAIQKMLDTPGPYLLDVIVPHQEHVLPMIPAGGGFSDVITEGDGRTKY

SEQ ID NO: 18 - protein, 668 aa, Borago officinalis
MASTPPSSTLTHPTTTPSSFPNHPKLFSSSFTLPFPVSPQTTSLSHSKHLRRHSLHPISNVISTRPSTSSPSSQNTPEQKEQLPFISRYAPNEPRKGADVLVEALERQGVTNVFAYPGGASMEIHQALTRSNIIKNVLPRHEQGGVFAAEGYARASGEPGVCIATSGPGATNLVSGLADALLDSVPMVAITGQVPRRMIGTDAFQETPIVEVTRSITKHNYLVLNVDDIPRIVKEAFYLARSGRPGPVLIDVPKDIQQQNVVPNWDVEMGLCGYISRLCKPPSELLLEQIVRLISEAKKPVLYVGGGCLNSSEELKRFVELTGIPVASTLMGLGSFPGSDELSLQMLGMHGTVYANYAVDKSDLMLAFGVRFDDRVTGKLEAFASRAKIVHIDIDPAEIGKNKQPHVSICADIKLALVGLNSILEKRAGNLKSNFKAWREELNEQKVKYPLTFKTFGDAIPPQYAIQTLDELTKGNAIITTGVGQHQMWAAQFYKYNRPRQWLTSAGLGAMGFGLPAAIGAVVARPDAVVVDIDGDGSFLMNVQELATIRVENLPVKIMLLNNQHLGMVVQWEDRFYKANRAHTYLGDPNHESEIFPDMLKFADACNIPAARVTKKHELGAAIQKMLDTPGPYLLDVIVPHQEHVLPMIPSGGTFDDVIVEGDGRTKY

SEQ ID NO: 19 - protein, 664 aa, Borago officinalis
MTATPHSSTLTHPTPTPTSFPSHPKLFSSSFTLPFPLSPQTTSLSHTKHIRRNSLHPISNVISPSPIPSSQSTPQQKQPPFISRYAPEEPRKGADVLVEALEREGVTNVFAYPGGASMEIHQALTRSNIIKNVLPRHEQGGVFAAEGYARASGDPGVCIATSGPGATNLVSGLADALLDSVPMVAITGQVPRRMIGTDAFQETPIVEVTRSITKHNYLVLSVDDIPRIVKEAFYLARSGRPGPVLIDVPKDIQQQMVVPHWDVEMGLSGYISRLCKPPCELLLEQIVRLISEAKRPVLYVGGGCLNSSEELKRFVELTGIPVASTLMGLGSFPGSDELSLQMLGMHGTVYANYAVDKSDLMLAFGVRFDDRVTGKLEAFASRAKIVHIDIDPAEIGKNKQPHVSICADIKLALAGLNSILEGRAGNLKANFSAWREELNEQKVKHPLTFKTFGDAIPPQYAIQTLDELTKGNAIISTGVGQHQMWAAQFYKYNRPRQWLTSAGLGAMGFGLPAAIGAVVARPDAVVVDIDGDGSFLMNVQELATIRVENLPVKIMLLNNQHLGMVVQWEDRFYKANRAHTYLGDPNHESEIFPDMLKFADACNIPAARVTKKNELRAAIQKMLDTPGPYLLDVVVPHQEHVLPMIPSGGTFDDVIVEGDGRTKY

SEQ ID NO: 20 - protein, 668 aa, Borago officinalis
MASTPPSSTLTHPTTTPSSFPNHPKLFSSSFTLPFPVSPQTTSLSHSKHLRRHSLHPISNVISTRPSTSSPSSQNTPEQKEQLPFISRYAPNEPRKGADVLVEALERQGVTNVFAYPGGASMEIHQALTRSNIIKNVLPRHEQGGVFAAEGYARASGEPGVCIATSGPGATNLVSGLADALLDSVPMVAITGQVPRRMIGTDAFQETPIVEVTRSITKHNYLVLNVDDIPRIVKEAFYLARSGRPGPVLIDVPKDIQQQNVVPNWDVEMGLCGYISRLCKPPSELLLEQIVRLISEAKKPVLYVGGGCLNSSEELKRFVELTGIPVASTLMGLGSFPGSDELSLQMLGMHGTVYANYAVDKSDLMLAFGVRFDDRVTGKLEAFASRAKIVHIDIDPAEIGKNKQPHVSICADIKLALVGLNSILEKRAGNLKSNFKAWREELNEQKVKYPLTFKTFGDAIPPQYAIQTLDELTKGNAIITTGVGQHQMWAAQFYKYNRPRQWLTSAGLGAMGFGLPAAIGAVVARPDAVVVDIDGDGSFLMNVQELATIRVENLPVKIMLLNNQHLGMVVQWEDRFYKANRAHTYLGDPNHESEIFPDMLKFADACNIPAARVTKKHELGAAIQKMLDTPGPYLLDVIVPHQEHVLPMIPNGGTFDDVIVEGDGRTKY

SEQ ID NO: 21 - DNA, 47 bp, Borago officinalis
gaaggtcggagtcaacggattgataacatcatcaaaggttccgccat

SEQ ID NO: 22 - DNA, 46 bp, Borago officinalis
gaaggtgaccaagttcatgctataacatcatcaaaggttccgccac

SEQ ID NO: 23 - DNA, 28 bp, Borago officinalis
cggaccatacttattggatgtcattgtc

SEQ ID NO: 24 - DNA, 2007 bp, Borago officinalis
atggcgtctactcctccttcctccaccctcacccaccccaccaccaccccctcctcatttcctaaccacccaaaactcttctcatcctccttcacccttccatttcctgtttccccccaaaccacctccctctcccactccaaacacctccgccgacattccctccacccaatctcaaacgtcatttccacccgtccttccacctcatctccctcttcccaaaatacccccgaacaaaaagaacaacttccattcatttccagatacgcccctaacgaaccaagaaaaggcgctgacgttctcgttgaagccctcgaaagacaaggagtgaccaacgtcttcgcctacccgggtggcgcctccatggagattcaccaagcgcttacccgctccaacattattaaaaacgtgcttcctaggcatgaacagggaggagtttttgcagctgagggatatgcacgtgcttcgggcgagccaggtgtttgtattgctacttctggacctggagcgacgaatcttgttagtggtttggctgatgctttgttggatagtgttcctatggtggcgattactggacaagtacctcgtaggatgattggtacggatgcttttcaagaaacgcctattgttgaggtaactaggtcgattaccaaacataattatcttgttttgaatgttgatgatattcctaggattgttaaggaagcgttttatttagcaaggagtggtaggcctggcccagttttgattgatgttcccaaagatattcagcaacagaatgtggttcctaattgggatgttgagatggggttgtgtggttatatttctaggttgtgtaagcctcctagtgaattgttgttggaacagattgtcaggttgatatctgaggccaaaaagcctgttctttatgtggggggagggtgtttgaattcgagtgaggagttgaagaggtttgttgagcttacggggattcctgtggcgagtactttgatggggttggggtcttttcctggttcagatgagttgtcgttgcagatgctggggatgcatgggactgtttatgcgaattatgctgtggataagagcgatttgatgcttgcatttggggttaggtttgatgaccgtgtgactgggaagttggaagcttttgctagtagggcgaagattgttcatattgatattgatcctgctgagattgggaagaacaagcagcctcatgtttcgatttgtgcagacattaagctggctttagtagggttgaattcaatattggagaagagagcggggaatttgaaatcaaatttcaaggcttggagggaggagctcaatgaacagaaggtgaaatatccgttgacgtttaaaacgtttggcgatgctattccaccacaatatgcaatccagactcttgatgaattgactaaggggaatgcaatcataaccacgggtgttggacaacatcagatgtgggctgctcagttttacaagtataatcgaccgcggcaatggttgacatcggctggattaggagccatgggttttggattgcctgctgctataggtgctgtggttgcaaggcctgatgccgttgttgtggatattgatggtgatggcagcttcctcatgaacgtccaggagttggcgactatccgtgtggagaatctcccagtcaaaataatgttgttaaataatcaacatttaggtatggtggtacagtgggaggatcgattctacaaggcgaatagagcacatacatatcttggagacccaaatcatgagtccgagatattcccagacatgttgaagtttgctgacgcctgtaatattcctgctgctcgagtgacaaagaagcatgaactgggagctgcaattcagaaaatgttagacacccccggaccatacttattggatgtcattgtcccacatcaagaacatgtgttgcctatgatcccaaatggcggaacctttgatgatgttatcgttgaaggtgatggaagaactaaatactaa

SEQ ID NO: 25 - DNA, 1995 bp, Borago officinalis
atgacggctactcctcattcatccaccctcactcaccccacccccacccccacctcatttcccagccacccaaaactcttctcctcctccttcaccctcccttttcccctttcaccccaaaccacctccctctcccataccaaacacatccgccgtaattctctccacccaatctcaaacgtcatttccccctctccaatcccctcttcccaaagtacccctcaacaaaaacaaccccccttcatttcaagatacgcccctgaagagccaagaaaaggagccgatgttctcgtggaagccttagaaagagaaggagtcaccaacgtcttcgcctacccgggtggcgcctctatggagatccatcaggccctcacccgctccaacattattaaaaacgtgcttcctagacatgaacagggtggtgttttcgcagctgagggatatgcacgagcttcgggcgacccgggtgtttgtattgctacttctggacccggtgcgacgaatcttgtaagtgggttggctgatgctttgttggatagtgtccctatggtggcgattactggacaagttcctcgtaggatgattggtactgatgcgtttcaagaaacacctattgttgaggtaactaggtctattactaaacataattatcttgttttgagtgttgatgatattcctaggattgttaaggaagcgttttatttagctaggagtggtaggcctggcccggttttgattgacgttcctaaagatattcagcaacagatggtggttcctcattgggatgttgagatggggttgagtggttatatttctaggttgtgtaagccgccttgtgaattgttgttggaacaaattgtgaggttgatttctgaggcgaaaaggccggtgctttatgtgggaggaggatgtttgaattcgagtgaggagttaaagaggtttgttgagcttacagggattcctgtggccagtactttgatgggtttggggtcatttcctggttcggatgagttgtcgttgcagatgctggggatgcatgggactgtttatgcgaattatgctgtggataagagtgatttgatgcttgcgtttggggttaggtttgatgatcgtgtgactgggaagttggaagcttttgctagtagggcaaagattgtccatattgatattgatcctgctgagattgggaagaacaagcagcctcatgtttcgatttgtgctgacattaagctggctttggcggggctgaattcgatattggaggggagagcggggaatttgaaagcaaatttctcggcttggagggaggagctcaatgaacagaaagtgaaacatccgttgacatttaaaacgtttggagatgctattccaccacaatatgcgattcagactcttgatgaattgactaaggggaatgcaatcataagtaccggtgttggacaacatcaaatgtgggcagctcagttttacaagtataatcgaccacggcaatggttgacgtcagctggattaggagccatgggatttggattgcctgctgctataggtgctgtggttgcaaggcctgatgccgttgttgtagatatagatggtgatggcagcttcctcatgaacgtgcaggagttggcgactattcgcgtggagaatctcccagtcaaaatcatgttgttaaataatcaacatttaggtatggtggtacagtgggaggaccgattctacaaggccaatagagcacatacatatcttggagatccaaatcatgagtccgagatattcccagacatgttgaagtttgctgacgcctgtaatattcctgctgctcgagtgacaaagaagaatgaactgagagctgcaatccagaaaatgttagacacccctggaccatacttattggatgtcgttgtgccacatcaagaacatgtgctgcctatgatcccaagtggcggaacctttgacgatgttattgttgaaggtgatggaagaactaaatactga

SEQ ID NO: 26 - DNA, 2007 bp, Borago officinalis
atggcgtctactcctccttcctccaccctcacccaccccaccaccaccccctcctcatttcctaaccacccaaaactcttctcatcctccttcacccttccatttcctgtttccccccaaaccacctccctctcccactccaaacacctccgccgacattccctccacccaatctcaaacgtcatttccacccgtccttccacctcatctccctcttcccaaaatacccccgaacaaaaagaacaacttccattcatttccagatacgcccctaacgaaccaagaaaaggcgctgacgttctcgttgaagccctcgaaagacaaggagtgaccaacgtcttcgcctacccgggtggcgcctccatggagattcaccaagcgcttacccgctccaacattattaaaaacgtgcttcctaggcatgaacagggaggagtttttgcagctgagggatatgcacgtgcttcgggcgagccaggtgtttgtattgctacttctggacctggagcgacgaatcttgttagtggtttggctgatgctttgttggatagtgttcctatggtggcgattactggacaagtacctcgtaggatgattggtacggatgcttttcaagaaacgcctattgttgaggtaactaggtcgattaccaaacataattatcttgttttgaatgttgatgatattcctaggattgttaaggaagcgttttatttagcaaggagtggtaggcctggcccagttttgattgatgttcccaaagatattcagcaacagaatgtggttcctaattgggatgttgagatggggttgtgtggttatatttctaggttgtgtaagcctcctagtgaattgttgttggaacagattgtcaggttgatatctgaggccaaaaagcctgttctttatgtggggggagggtgtttgaattcgagtgaggagttgaagaggtttgttgagcttacggggattcctgtggcgagtactttgatggggttggggtcttttcctggttcagatgagttgtcgttgcagatgctggggatgcatgggactgtttatgcgaattatgctgtggataagagcgatttgatgcttgcatttggggttaggtttgatgaccgtgtgactgggaagttggaagcttttgctagtagggcgaagattgttcatattgatattgatcctgctgagattgggaagaacaagcagcctcatgtttcgatttgtgcagacattaagctggctttagtagggttgaattcaatattggagaagagagcggggaatttgaaatcaaatttcaaggcttggagggaggagctcaatgaacagaaggtgaaatatccgttgacgtttaaaacgtttggcgatgctattccaccacaatatgcaatccagactcttgatgaattgactaaggggaatgcaatcataaccacgggtgttggacaacatcagatgtgggctgctcagttttacaagtataatcgaccgcggcaatggttgacatcggctggattaggagccatgggttttggattgcctgctgctataggtgctgtggttgcaaggcctgatgccgttgttgtggatattgatggtgatggcagcttcctcatgaacgtccaggagttggcgactatccgtgtggagaatctcccagtcaaaataatgttgttaaataatcaacatttaggtatggtggtacagtgggaggatcgattctacaaggcgaatagagcacatacatatcttggagacccaaatcatgagtccgagatattcccagacatgttgaagtttgctgacgcctgtaatattcctgctgctcgagtgacaaagaagcatgaactgggagctgcaattcagaaaatgttagacacccccggaccatacttattggatgtcattgtcccacatcaagaacatgtgttgcctatgatcccaagtggcggaacctttgatgatgttatcgttgaaggtgatggaagaactaaatactaa

SEQ ID NO: 27 - DNA, 1995 bp, Borago officinalis
atgacggctactcctcattcatccaccctcactcaccccacccccacccccacctcatttcccagccacccaaaactcttctcctcctccttcaccctcccttttcccctttcaccccaaaccacctccctctcccataccaaacacatccgccgtaattctctccacccaatctcaaacgtcatttccccctctccaatcccctcttcccaaagtacccctcaacaaaaacaaccccccttcatttcaagatacgcccctgaagagccaagaaaaggagccgatgttctcgtggaagccttagaaagagaaggagtcaccaacgtcttcgcctacccgggtggcgcctctatggagatccatcaggccctcacccgctccaacattattaaaaacgtgcttcctagacatgaacagggtggtgttttcgcagctgagggatatgcacgagcttcgggcgacccgggtgtttgtattgctacttctggacccggtgcgacgaatcttgtaagtgggttggctgatgctttgttggatagtgtccctatggtggcgattactggacaagttcctcgtaggatgattggtactgatgcgtttcaagaaacacctattgttgaggtaactaggtctattactaaacataattatcttgttttgagtgttgatgatattcctaggattgttaaggaagcgttttatttagctaggagtggtaggcctggcccggttttgattgacgttcctaaagatattcagcaacagatggtggttcctcattgggatgttgagatggggttgagtggttatatttctaggttgtgtaagccgccttgtgaattgttgttggaacaaattgtgaggttgatttctgaggcgaaaaggccggtgctttatgtgggaggaggatgtttgaattcgagtgaggagttaaagaggtttgttgagcttacagggattcctgtggccagtactttgatgggtttggggtcatttcctggttcggatgagttgtcgttgcagatgctggggatgcatgggactgtttatgcgaattatgctgtggataagagtgatttgatgcttgcgtttggggttaggtttgatgatcgtgtgactgggaagttggaagcttttgctagtagggcaaagattgtccatattgatattgatcctgctgagattgggaagaacaagcagcctcatgtttcgatttgtgctgacattaagctggctttggcggggctgaattcgatattggaggggagagcggggaatttgaaagcaaatttctcggcttggagggaggagctcaatgaacagaaagtgaaacatccgttgacatttaaaacgtttggagatgctattccaccacaatatgcgattcagactcttgatgaattgactaaggggaatgcaatcataagtaccggtgttggacaacatcaaatgtgggcagctcagttttacaagtataatcgaccacggcaatggttgacgtcagctggattaggagccatgggatttggattgcctgctgctataggtgctgtggttgcaaggcctgatgccgttgttgtagatatagatggtgatggcagcttcctcatgaacgtgcaggagttggcgactattcgcgtggagaatctcccagtcaaaatcatgttgttaaataatcaacatttaggtatggtggtacagtgggaggaccgattctacaaggccaatagagcacatacatatcttggagatccaaatcatgagtccgagatattcccagacatgttgaagtttgctgacgcctgtaatattcctgctgctcgagtgacaaagaagaatgaactgagagctgcaatccagaaaatgttagacacccctggaccatacttattggatgtcgttgtgccacatcaagaacatgtgctgcctatgatcccaaatggcggaacctttgacgatgttattgttgaaggtgatggaagaactaaatactga

SEQ ID NO: 28 - protein, 664 aa, Borago officinalis
MTATPHSSTLTHPTPTPTSFPSHPKLFSSSFTLPFPLSPQTTSLSHTKHIRRNSLHPISNVISPSPIPSSQSTPQQKQPPFISRYAPEEPRKGADVLVEALEREGVTNVFAYPGGASMEIHQALTRSNIIKNVLPRHEQGGVFAAEGYARASGDPGVCIATSGPGATNLVSGLADALLDSVPMVAITGQVPRRMIGTDAFQETPIVEVTRSITKHNYLVLSVDDIPRIVKEAFYLARSGRPGPVLIDVPKDIQQQMVVPHWDVEMGLSGYISRLCKPPCELLLEQIVRLISEAKRPVLYVGGGCLNSSEELKRFVELTGIPVASTLMGLGSFPGSDELSLQMLGMHGTVYANYAVDKSDLMLAFGVRFDDRVTGKLEAFASRAKIVHIDIDPAEIGKNKQPHVSICADIKLALAGLNSILEGRAGNLKANFSAWREELNEQKVKHPLTFKTFGDAIPPQYAIQTLDELTKGNAIISTGVGQHQMWAAQFYKYNRPRQWLTSAGLGAMGFGLPAAIGAVVARPDAVVVDIDGDGSFLMNVQELATIRVENLPVKIMLLNNQHLGMVVQWEDRFYKANRAHTYLGDPNHESEIFPDMLKFADACNIPAARVTKKNELRAAIQKMLDTPGPYLLDVVVPHQEHVLPMIPNGGTFDDVIVEGDGRTKY

(19) United States
(12) Patent Application Publication
(10) Pub. No.: US 2017/0298380 A1
(43) Pub. Date: Oct. 19, 2017

(54) IMIDAZOLINONE HERBICIDE RESISTANT BORAGE

(71) Applicant: Bioriginal Food & Science Corp., Saskatoon (CA)

(72) Inventors: Guohai Wu, Saskatoon (CA); Dongyan Song, Saskatoon (CA); Patricia Vrinten, Saskatoon (CA); Xiao Qiu, Saskatoon (CA)

(73) Assignee: Bioriginal Food & Science Corp., Saskatoon (CA)

(21) Appl. No.: 15/098,191

(22) Filed: Apr. 13, 2016

(51) Int. Cl. C12N 15/82; A01N 43/50

(52) U.S. Cl. CPC C12N 15/8274; A01N 43/50

(57) ABSTRACT

The present disclosure provides borage plants having increased resistance to imidazolinone herbicides. More particularly, provided herein are methods for generating herbicide resistant borage and testing of selected progeny for homozygosity. Nucleic acids encoding AHAS 1 and 2 genes that encode herbicide resistance in borage are provided.

### REFERENCE TO SEQUENCE LISTING SUBMITTED ELECTRONICALLY

The Sequence Listing associated with the application is provided in text file format in lieu of a paper copy, and is hereby incorporated by reference into the specification. The name of the text file containing the Sequence Listing is SequenceListing_ST25.txt. The text file is 48 kilobytes, was created on Apr. 26, 2016 and is submitted electronically via EFS-Web.

### FIELD OF THE INVENTION

This invention relates generally to methods for generation of herbicide resistant plants and more specifically to herbicide resistant borage plants.

### BACKGROUND OF THE INVENTION

Borage (Borago officinalis L.) is an annual herb from Boraginaceae family that is native to the Mediterranean region but has now naturalized worldwide. However, it is mainly grown commercially in the United Kingdom, the Netherlands, Canada, New Zealand and Poland, which currently account for 95% of global production. Historically, borage has been used for culinary and medicinal purposes. Recently, borage oil has gained attention and interest from medical and nutritional research due to its high content of gamma-linolenic acid (GLA).

GLA has been utilized for both anti-inflammatory and anti-cancer actions purposes and has been shown to be effective in treating diabetic neuropathy leading to improved blood flow and reduced tingling of extremities. In addition, a number of studies investigating the role of GLA in cardiovascular health have suggested that dietary GLA reduces low-density lipoprotein cholesterol, plasma triacylglycerols, blood pressure and smooth muscle proliferation. In light of the efficacy of GLA in treating physiological disorders and diseases caused by deficiencies in essential fatty acids and anti-inflammatory secondary messengers, several sources of GLA including borage oil have been developed.

Borage is well known for rich GLA content ranging from 16% to 28% in the seed oil and its total oil content in a seed can reach 27% to 37% (w/w). The variation in GLA content is contributed by multiple factors including geographical location, length of the light period during growing season, average temperature and diurnal temperature difference. Besides GLA, borage oil also contains a significant amount of linoleic acid (LA), one of essential omega-6 fatty acids, up to 38%, but there is no relationship between the content of LA and GLA.

Borage oil containing GLA has shown positive effect in treating a number of clinical conditions caused by GLA deficiency in humans. In addition, a study also suggests that consumption of borage oil can improve fatty acid metabolism and skin function in elderly people. Brosche T, Platt D. “Effect of borage oil consumption on fatty acid metabolism, transepidermal water loss and skin parameters in elderly people” Archives of Gerontology and Geriatrics 30 (2000) 139-50. Because of its positive properties, many nutraceutical supplements, food products and body-care products have now been enriched with borage oil, resulting in a surge of demand for borage farming. Besides high oil content and rich GLA level, large seed size of borage also makes harvest and oil extraction much easier thereby making borage the most preferred source of GLA in comparison to other plants.

The yields and quality of borage cultivation are determined by many factors including weed management. Weeds are the major threat to the production of many crops and cause losses in the billions of dollars. Due to lack of an herbicide resistant variety, weeding for borage still primarily relies on hand laborers and is constantly required at least until flowering when the plants are well established. Thus, weeding requirements notably increase the cost of borage production with the result that fewer farmers are willing to cultivate borage in a large scale. From the foregoing, it appeared to the present inventors that herbicide resistant borage plants were needed.

### BRIEF SUMMARY OF THE INVENTION

Provided herein is a description of the creation, identification and characterization of chemcially induced borage mutants selected for resistance to the herbicide imidazolinone. An EMS-mutagenized borage population was generated by using a series of concentrations of EMS to treat M1 seeds. After screening M2 borage plants with the herbicide, tolerant plants were selected, self-pollinated and grown to their maturity. The offspring were subjected to herbicide screening again to confirm the phenotype, resulting in identification of two genetically stable imidazolinone-resistant lines.

Two acetohydroxyacid synthase (AHAS) genes, AHAS1 and AHAS2, involved in the imidazolinone resistance were isolated and sequenced from both mutant (resistant) and wild type (susceptible) borage plants. Comparison of these AHAS sequences revealed that a single nucleotide substitution occurred in the AHAS1 resulting in an amino acid change from serine (S) in the susceptible plant to asparagine (N) in the first resistant line. The similar substitution was later found in the AHAS2 of the second resistant line.

A KASP marker was developed for the AHAS1 mutation to differentiate the homozygous susceptible, homozygous and heterozygous resistant borage plants for the breeding purpose. An in vitro assay showed homozygous resistant borage containing the AHAS1 mutation could retain significantly higher AHAS activity than susceptible borage across different imazamox concentrations. The herbicide dose response test showed that the resistant line with the AHAS1 mutation was tolerant to four times the field applied concentration of the “Solo” herbicide as a representative Type 2 herbicide.

### BRIEF DESCRIPTION OF THE DRAWINGS

For a more complete understanding of the present invention, including features and advantages, reference is now made to the detailed description of the invention along with the accompanying figures:

FIG. 1 shows a picture of the first tolerant borage plant (an AHAS1 mutant) from offspring of M1 seeds that were treated with 1.5% EMS for 16 hours.

FIG. 2 shows a picture of the second imidazolinone tolerant borage plant (an AHAS2 mutant) identified by herbicide treatment of the offspring of M4 seeds.

FIG. 3 shows a protein sequence comparison of borage and other plants showing that borage AHAS1 and AHAS2 proteins were 95% identical at the amino acid level and both shared approximately 75% of amino acid identity with Arabidopsis AHAS protein and 80% with the sunflower AHAS1 protein sequence.

FIG. 4 presents an alignment of partial sequence of AHAS1 and AHAS2 from imidazolinone susceptible and resistant borage. The top two sequences are AHAS genes of susceptible borage. The bottom two sequences are mutant AHAS1 and AHAS2 from two resistant borage lines. Single nucleotide substitutions from G to A in mutant AHAS1 at 1953 bp and mutant AHAS2 at 1941 bp were highlighted in the dashed line box.

FIGS. 5A and 5B represent an alignment of AHAS protein sequences from Arabidopsis thaliana, sunflower (Helianthus annuus) and both susceptible and resistant borage lines. FIG. 5B is a continuation of the sequences depicted in FIG. 5A. The completely and partially identical amino acids were highlighted. Amino acid substitutions in AHAS1 and AHAS2 of two separate tolerant lines were highlighted in the dashed box.

FIG. 6 represents KASP genotyping plot for M3 borage plants. Samples marked red are the homozygous resistant for the FAM allele (solid oval), blue are the homozygous susceptible for the HEX allele (dashed oval) and green are the heterozygous (dotted oval); “X” are two no-template controls.

FIG. 7A shows the results of a comparison of specific AHAS activities between the AHAS1 mutant and wild type across different imazamox concentrations. The activity at 0 μM imazamox was as 100%; the same letter means that the activities are not significantly different (P >0.05). FIG. 7B shows the data and statistical analysis of the results depicted in FIG. 7A. Means with the same letter in the same column are not significantly different (P >0.05). The multi-treatment comparisons is using the Tukey method. SEM=standard error of mean. Genotypes, herbicide concentrations and the interaction of genotype variety(G)*herbicide (H) concentrations all showed significant effects on AHAS enzyme activity because their P values are less than 0.05.

FIGS. 8A-D depict the herbicide dosage response test showing that homozygous resistant borage tolerated up to 4X “Solo” herbicide. The tray depicted in FIG. 8A was wild-type borage control without herbicide treatment; the tray depicted in FIG. 8B was homozygous resistant borage treated with 2X “Solo”; the tray depicted in FIG. 8C was homozygous resistant borage treated with 4X “Solo”; the tray depicted in FIG. 8D was wild-type borage control treated with 2X “Solo”. The image was taken at 21 days after the treatment.

FIG. 9 shows that the M4 homozygous resistant borage showed strong resistance to herbicide “Solo” and “Pursuit”, and it also exhibited moderate tolerance towards “Everest 2.0”. From left to right: mutant borage treated with 2X “Solo”; mutant borage treated with 2X “Pursuit”; mutant borage treated with 2X “Everest 2.0”. The image was taken at 21 days after the treatment.

FIG. 10 shows the Borago officinalis Wild Type (Imidazolinone Susceptible) AHAS1 Nucleotide Sequence.

FIG. 11 shows the Borago officinalis Wild Type (Imidazolinone Susceptible) AHAS2 Nucleotide Sequence.

FIG. 12 shows the Borago officinalis Mutant (Imidazolinone Resistant) AHAS1 Nucleotide Sequence.

FIG. 13 shows the Borago officinalis Mutant (Imidazolinone Resistant) AHAS2 Nucleotide Sequence.

### DETAILED DESCRIPTION OF THE INVENTION

In one certain embodiments herein described, herbicide resistant borage varieties were developed. Involved in this development were methods of testing chemically induced mutants that exhibited herbicide resistance in order to identify heterozygosity and more rapidly develop varieties that were homogenous for the induced herbicide resistance. Exemplary of this process ethyl methanesulfonate (EMS) induced borage mutants were created, identified and characterized for imidazolinone resistance. An EMS-mutagenized borage population was generated by using a series of concentrations of EMS to treat M1 seeds. After screening M2 borage plants with the herbicide, tolerant plants were selected, self-pollinated and grown to their maturity. The offspring were subjected to herbicide screening again to confirm the phenotype, resulting in identification of two genetically stable imidazolinone-resistant lines. Two acetohydroxyacid synthase (AHAS) genes, AHAS1 and AHAS2, involved in the imidazolinone resistance were isolated and sequenced from both mutant (resistant) and wild type (susceptible) borage plants. Comparison of these AHAS sequences revealed that a single nucleotide substitution occurred in the AHAS1 resulting in an amino acid change from serine (S) in the susceptible plant to asparagine (N) in the first resistant line. The similar substitution was later found in the AHAS2 of the second resistant line. A KASP marker was developed for the AHAS1 mutation to differentiate homozygous susceptible, homozygous and heterozygous resistant borage plants for the breeding purpose. An in vitro assay showed homozygous resistant borage containing the AHAS1 mutation could retain significantly higher AHAS activity than susceptible borage across different imazamox concentrations. The herbicide dose response test showed that the resistant line with the AHAS1 mutation was tolerant to four times the field applied concentration of the SOLO brand herbicide.

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

Borage is an erect and hispid plant that can normally grow up to 60-100 cm (2.0-3.3 ft) in height; and it has simple alternate leaves that are obovate, ovate or oblong with an obtuse apex. The stem is cylindrical, hollow and succulent. The stems, leaves and calyx are covered with stiff unicellular trichomes. Borage is also known as “star flower” because of the shape of the flower. The flowers vary in color including bright blue, violet, and pink, even white at different stages and between biotypes. Borage flowers are produced on scorpioid cymes which arise from the axils of the leaves at intervals on the stem. Flowering proceeds basipetally in the inflorescence and each inflorescence develops several flowers. Each flower contains a deeply 4-lobed ovary in gynobasic style. As the flower matures, it develops into 3-4 ovoid or oblong seeds. The seed coat will develop color from green to brown and then black signifying maturity. After then, the seed will abscise in short order although varieties have been developed with the trait of “seed retention”. Borage is an allogamous plant with an entomophilous pollination system, meaning that insects such as bees take pollen grains and spread them onto neighboring flowers. As with all cultivated plants, competition with weeds severely affects productively of borage. Because herbicide resistant borage has not been available, hand weeding is utilized in borage cultivation and the requirement for hand weeding has significantly impacted the commercial production of borage.

Herbicides represent a large array of chemical compounds able to kill weed plant species. They usually act at targeted sites of essential enzymes where metabolic function and energy transfer are taking place in plant cells, thereby inhibiting the enzymatic function. More than 60% of herbicides introduced in the last four decades are designed to interfere with the function of chloroplasts in plants, even though the action mechanism of some commercial herbicides is not yet fully clear. Based on modes of action, commercial herbicides are classified into 27 groups (Alberta Agriculture and Rural Development, 2014). Among them, the mechanism of action of the group 2 herbicides falls into the category of inhibition of amino acid biosynthesis, specifically by inhibiting the enzyme acetohydroxyacid synthase (AHAS), also known as acetolactate synthase (ALS).

AHAS catalyzes the first reaction in the pathway for synthesis of the branched chain amino acids leucine, isoleucine and valine in plants and many microorganisms. An unusual feature of the pathway is two parallel condensation reactions catalyzed by the AHAS enzyme leading to the formation of valine and isoleucine: two pyruvate molecules produce carbon dioxide (CO₂) and 2-acetolactate—a precursor of valine and leucine, while one pyruvate molecule and α-ketobutyrate form CO₂ and 2-acetohydroxybutyrate—a precursor of isoleucine.

Due to the critical role in ensuring a balanced supply of the amino acids as well as producing intermediates to interact with other cellular metabolic pathways, AHAS enzyme activity is carefully regulated by various mechanisms. One of the mechanisms regulating AHAS activity by end-product feedback inhibition is carried out by the regulatory subunit of AHAS enzyme. Almost all AHAS can be inhibited by at least one of the branched chain amino acids, and valine is clearly the most potent inhibitor in microorganisms and plants. Leucine is an equally good or sometimes better inhibitor than valine. The other mechanism involves the control of the enzyme at the transcriptional level. In plants, at least one AHAS gene is expressed in a constitutive manner but the expression level may vary between tissues and developmental stages. The highest level of AHAS transcription and activity is observed in the metabolically active meristematic tissues. Some plants possess multiple AHAS genes, two of which are housekeeping and other AHAS genes are only expressed in a tissue specific manner. AHAS genes have been identified and sequenced in a variety of plants and microorganisms. In 2000, Duggleby and Pang identified up to 73 conserved residues in the overall alignment of 24 AHAS sequences from various organisms. Duggleby R G and Pang S S. “Acetohydroxyacid Synthase” Journal of Biochemistry and Molecular Biology 33 (2000) 1-36. However, the function of these residues has never been directly tested or fully understood except being deduced by analogy with related enzymes.

Interestingly enough, although AHAS sequences from different species share a large number of amino acid identities, AHAS sequences from plants and some fungi are observed to be substantially longer than other microorganisms due to an N-terminal extension. The AHAS enzyme is normally located in plastids for plants or mitochondria for fungi; that is, it must be transported to these organelles after the enzyme is synthesized. Therefore, N-terminal extension is probably involved in the intracellular trafficking of an AHAS enzyme. See Duggleby and Pang (2000), supra.

Also, amino acid composition of the N-terminal extension with a high number of serine residues is a typical feature of chloroplast and mitochondrial transit peptides. The function of the transit peptide is to guide the protein to the target organelle and it is cleaved off during or after translocation. However, the actual cleavage site has not been established for any AHAS protein. Some experimental evidence indicates that the N-terminal extension is non-essential for AHAS activity but removing part or all of the transit peptide sequence has been shown to be crucial for expression of plant AHAS enzyme in a recombinant system in microorganisms. Plant AHAS genes are expected to encode polypeptides with a molecular mass of about 72 kDa, which is roughly 10 kDa larger than bacterial AHAS catalytic subunits. However, the mature AHAS protein with only 65 kDa mass or less is found in a variety of monocotyledonous and dicotyledonous plant species suggesting that the extra 10 kDa is possibly contributed by the N terminal organelle targeting sequence.

The AHAS enzyme requires thiamine diphosphate (ThDP), flavin adenine dinucleotide (FAD) and a divalent metal ion as cofactors to catalyze the initial decarboxylation of pyruvate. ThDP is essential for AHAS activity from all species. All ThDP-dependent enzymes contain a conserved 29-32 amino acid motif that begins with the triplet amino acids GDG and ends with NN to interact with ThDP. With no exception, AHAS enzymes also contain exactly the same motif. The role of ThDP is to break the bond between keto group and carboxyl group carbons of pyruvate to form an intermediate product. The intermediate condenses with the 2-ketoacid substrate to the end product while ThDP is regenerated. FAD is required for AHAS activity, but its role is not fully understood yet. Two hypotheses of FAD's role have been proposed: the first is that FAD supports the structure of the enzyme in order to maintain the correct geometry for substrate binding and catalytic activity. The second hypothesis is that FAD protects α-caranion from protonation during the binding process of 2-ketoacid substrate by allowing the enamine to form a reversible adduct with FAD.

Metal ions are commonly required by all ThDP-dependent enzymes including AHAS for activity, and the requirement is generally satisfied with Mg2+. The role of the metal ion is to act as an anchor to hold the ThDP in place by coordinating it to two of the phosphate oxygen atoms from ThDP and two amino acid side chains from the ThDP-motif of an AHAS. These cofactors are essential for AHAS activity, so they are also required for enzymatic assays of AHAS activity.

In most of the studies on AHAS, the enzyme activity is measured using a discontinuous colorimetric assay. In the method, the sample containing AHAS enzyme is incubated for a fixed time between 30 minutes to 2 hours with pyruvate and other additives (including those cofactors). ThDP is included at a concentration of 50 μM at least or more; the metal ion is usually required at a concentration of 0.1 to 10 mM; and FAD is added at a concentration of 2 to 100 μM. The reaction is then terminated by adding sulfuric acid and heated at 60° C. for 15 minutes to convert acetolactate to acetoin. By reacting with creatine and α-naphthol, acetoin is converted to a pink-colored complex which can be measured at 520 nm wavelength in a spectrometer. As a result, AHAS activity can be estimated based on the color intensity. In order to maintain high activity and stability of the enzyme during series of treatments in an assay, a high concentration of potassium phosphate is recommended at optimal pH 7.0-7.5 in the extraction buffer. In addition, high concentrations of glycerol and polyvinylpolypyrolidone (PVPP) have also been reported to help with stabilization of the enzyme in the assay. An advantage of the method is the excellent sensitivity with ability to measure as low as 0.0001 units of the enzyme activity routinely.

Group 2 herbicides consist of five chemical families including imidazolinones, sulfonylureas, triazolopyrimidines, pyrimidinylthiobenzoates and sulfonylamino-carbonyltriazolinones. See Mallory-Smith C A and Retzinger E J Jr. “Revised classification of herbicides by site of action for weed resistance management strategies” Weed Technology 17 (2003) 605-619.

Among different groups of herbicides, imidazolinone herbicide controls a broad spectrum of weeds at a relatively low application rate. Imidazolinones include imazapyr, imazapic, imazethapyr, imazamox, imazamethabenz and imazaquin (Table 1), and all of them contain an imidazole moiety in the molecular structure.

TABLE 1
Imidazolinone herbicides

| | | Exemplary |
|---|---|---|
| Generic name: | IUPAC Chemical Name: | Tradenames: |
| imazamox | 2-[4,5-dihydro-4-methyl-4-(1-methylethyl)-5-oxo- | RAPTOR |
| | 1H-imidazol-2-yl]-5-(methoxymethl)-3- | |
| | pyridinecarboxylic acid. | |
| imazapic | 2-[4,5-dihydro-4-methyl-4-(1-methylethyl)-5-oxo- | CADRE |
| | 1H-imidazol-2-yl]-5-methyl-3-pyridinecarboxylic | |
| | acid | |
| imazapyr | 2-(4,5-Dihydro-4-methyl-4-(1-methylethyl)-5-oxo- | ARSENAL |
| | 1H-imidazol-2-yl)-3-pyridine carboxylic acid | |
| imazaquin | 2-(4-methyl-5-oxo-4-propan-2-yl-1H-imidazol-2-yl) | SCEPTER |
| | quinoline-3-carboxylic acid | |
| imazethapyr | 2-[4.5-dihydro-4-methyl-4-(1-methylethyl)-5-oxo- | PURSUIT |
| | 1H-imidazol-2-yl]-5-ethyl-3-pyridinecarboxylic acid | |
| imazmethabenz- | methyl 4-methyl-2-(4-methyl-5-oxo-4-propan-2-yl- | ASSERT, |
| methyl | 1H-imidazol-2-yl)benzoate | DAGGER |
| imazapyr/imazamox | | ODYSSEY |
| mixture | | |

Based on the second cyclic structure of the molecules excluding the imidazole ring, they can be further divided into three groups: pyridine imazolinones, benzene imazolinone, and quinolone imazolinone as shown below.

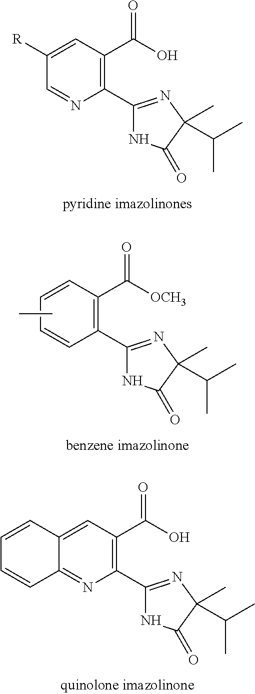

As shown above, imazamethabenz contains a benzene ring, imazaquin has a quinoline moiety and the rest of imidazolinones are characterized by a pyridine ring. In the pyridine imazolineones, analogues are differentiated by the R-group of the pyrimidine ring. Thus in imazapyr: R═H, in imazapic: R═CH₃, in imazethapyr: R═CH₃—CH₂, and in imazamox: R═CH₃—O—CH₂. Despite the chemical differences, the activity of AHAS inhibition among pyridine imidazolinones is very similar and may be related to certain characteristics of the metabolism of pyridine imidazolinones in plants. Besides the strong link between imidazole ring and AHAS inhibition, the second cyclic structures, pyridine, benzene and quinoline rings, also play important role in AHAS inhibition resulting in the different inhibition activities. See Tan S, et al “Imidazolinone-tolerant crops: history, current status and future” Pest Management Science 61 (2005) 246-257. Imidazolinone herbicides are effective to control a wide spectrum of grass and broadleaf weeds at a low application rate with low mammalian toxicity and have many ideal traits for utilization in developing an herbicide resistant crop.

After the discovery of a variety of imidazolinone tolerant plant species with altered AHAS genes, a number of imidazolinone resistant crops have been developed through traditional breeding and transgenic approaches as well as mutagenesis and selection. See e.g. U.S. Pat. No. 7,829,762.

In reference to Arabidopsis thaliana L., five commonly occurring mutations in the AHAS catalytic subunit at Ala122, Pro197, Ala205, Trp574 and Ser653 have been found to contribute to tolerance to AHAS inhibitors. See Christoffers M J, et al. “Altered herbicide target sites: implications for herbicide-resistant weed management” In: Inderjit (ed) Weed biology and management. Kluwer Academic, Dordrecht (2004) 199-210; Tranel P J, Wright T R. “Resistance of weeds to ALS-inhibiting herbicides: what have we learned?” Weed Science 50 (2002) 700-712; and Tan (2005), supra. Tolerance is conferred by these mutations because they are located closely within the adjacent area of the protein to form a pocket where the binding site of AHAS inhibitors is located. Based on molecular modeling of the interaction between AHAS and imidazolinones, the binding pocket is believed as the entry site of the substrate for an AHAS enzyme. Thereby, once imidazolinones enter the substrate access channel, they will impede the binding of the substrate to AHAS resulting in loss of activity.

Among the five common mutations, Trp574 mutation leads to tolerance to all families of group 2 herbicides; mutation at Pro197 is only tolerant to sulfonylureas and mutations at Ala122, Ala205 and Ser653 are more tolerant to imidazolinones. The Ser653 mutation confers strong tolerance to imidazolinones, but not cross-tolerance to other chemical families in group 2 herbicides, which is preferable for the development of imidazolinone resistant crops.

Chemically Induced Mutagenesis:

Induced mutagenesis seeks to induce changes in chromosomal DNA more rapidly than happens in nature. Both irradiation and chemical mutagenesis may be employed. As an exemplary but non-limiting mutagen, ethyl methanesulfonate (EMS) was utilized to generate herbicide resistant borage in the examples provided herein. Alternative mutagens that are employed in plant mutagenesis include irradiation (formerly X-ray, but now more typically fast neutron or gamma ray bombardment), treatment with sodium azide (Az), methylnitrosourea (MNU), the combination Az-MNU, diethyl sulfate (DES) and diepoxybutante (DEB), all of which induce DNA mutations. Among these irradiation is more likely to cause deletions and translocations while the chemical mutagens are more likely to cause single base-pair (bp) changes, or single-nucleotide polymorphisms (SNPs). EMS selectively alkylates guanine bases resulting in mismatching and bases changes during DNA synthesis, typically resulting in GC to AT basepair transitions. In contrast, Az-MNU can cause either GC to AT or AT to GC transitions.

In one embodiment, borage was chemically mutagenized with EMS to generate a population to be selected for the desired herbicide resistant trait. In order to be efficient in producing a mutant population, EMS mutagenesis must reach an optimized balance of relatively high mutation rate and minimized sterility in M1 (EMS-treated seeds) and M2 (M1 offspring) generations. However, it is difficult to achieve the balance because of limited information available for EMS concentration and the lethal dose for different plant species. Normally high dosage causes a strong mutation rate; but it also increases unwanted mutations on various loci leading to a high rate of sterility or even lethality. To determine the efficacy of chemical mutagenesis, two criteria must be considered: the ratio of sterility, and pigment defects in M1 plants. The sterility of M1 plants is supposed to be significant after an effective treatment, that is, 20-50% of M1 plants should have no offspring. The pigment defect ratio should be up to 1% of M1 plants according to some experts. In addition to the mutagen dose, the duration of chemical treatment is another factor affecting chemical mutagenesis. Although the mutation occurs to M1 plants, the mutant phenotype may not be shown because most mutations are genetically recessive and M1 generation is usually heterozygous for the mutations. Self-pollination of M1 plants is required to produce M2 seeds, in which heterozygous mutations will segregate resulting in variations in mutant phenotypes. See Koornneef M “Classical mutagenesis in higher plants.” In: Molecular Plant Biology, Vol. 1 (2002) pp. 1-11. Oxford University Press. Therefore, M2 borage plants were used to screen for imidazolinone resistance.

The following examples are include for the sake of completeness of disclosure and to illustrate the methods of making the compositions and composites of the present invention as well as to present certain characteristics of the compositions. In no way are these examples intended to limit the scope or teaching of this disclosure.

### Example 1

### Chemically Induced Mutagenesis of Borage

Ethyl methanesulfonate (EMS), a chemical mutagen, can induce nucleotide mismatching and base changes in a genome resulting in genetic mutations. Under the effect of EMS, guanine (G) undergoes alkylation to form O6-ethylguanine, which prefers thymine (T) to cytosine (C) pairing during DNA synthesis; thereby the original G/C pair is replaced by A (adenine)/T pair. The nucleotide substitution could lead to changes of amino acids at critical positions resulting in sensitivity variations to herbicides. Although the mutation occurs to M1 plants, mutant phenotypes may or may not be shown in the M1 generation because most mutations are genetically recessive. Self-pollination of M1 to produce M2 is necessary to allow heterozygous mutants to segregate resulting in variations in mutant phenotypes, thus M2 plants are screened for herbicide resistance. EMS mutagenesis has been used to produce imidazolinone resistant Arabidopsis and other plant species through induced mutations in AHAS genes.

Efforts were undertaken to develop imidazolinone herbicide resistant borage through EMS mutagenesis and herbicide screening. Different concentrations of EMS were used to treat M1 borage seeds and the mutagenized seeds were then grown to maturity in the field to obtain M2 seeds. M2 plants were screened for imidazolinone tolerance and surviving individuals were selected and self-pollinated manually to produce M3 seeds. The M3 plants were subjected to herbicide screening again to confirm the phenotype. This process resulted in identification of two stable imidazolinone resistant lines at different phases of the project.

Generation of an EMS Mutagenized Borage Population.

Approximately 164000 borage seeds were divided into 6 groups for mutagenesis. The seeds (M1) were soaked in 0.5%, 1.0% and 1.5% (v/v) of EMS solutions for 8 hours and 16 hours respectively, and then rinsed with tap water for 4 hours. After washing, the seeds are dried with paper towel. The mutagenized seeds were sowed in 48 plots in a research farm in Saskatoon, SK, in June, 2012. Each plot was 7.5×1.5 m in size and sowed 86 g of mutagenized seeds. M1 borage plants were grown to maturity and M2 seeds were harvested by groups according to the EMS treatments.

Herbicide Tolerance Screening of Mutagenized M2 Population and Wild-Type Population.

Herbicide tolerance screening was carried out in a growth chamber. M2 seeds were planted at 1-2 cm in 25×50 cm flats containing commercial potting mix (Sunshine Mix 3; Sun Gro.) in the growth chamber under a 16 hour light (22° C.) and 8 hour dark (16° C.) cycle. Each flat contained 72 seeds. Group 2 herbicide, “Solo” (BASF Corp.), was applied over foliage when most plants were at the two-leaf stage in a specialized herbicide treatment chamber. The spray solution included 84 g ai/ha (active ingredient/per hectare) imazamox with adjuvant Merge (BASF Corp.) at 0.5% (v/v). A moving nozzle cabinet sprayer with a flat-fan nozzle tip was calibrated to deliver 102 L/ha spray solution in a single pass. M2 plants were visually evaluated 21 days after herbicide spray by comparing herbicide treated and untreated wild-type borage controls. Putative tolerant M2 plants were transplanted, self-pollinated and grown to maturity. Their offspring, M3, underwent the same screening process to confirm imidazolinone-resistant phenotype. False positive materials were discarded, and truly tolerant materials were archived. The screening process was carried out continuously until homozygous resistant plants were identified.

Wild-Type Borage was Also Screened for Imidazolinone Resistance.

Approximately 14 kg wild-type seeds were sowed in the field in a research farm in Saskatoon, SK, in June, 2013. “Solo” herbicide containing 84 g ai/ha imazamox with adjuvant Merge at 0.5% (v/v) was applied over foliage by a tractor sprayer at a spray rate of 100 L/ha, when most plants were at the 2-4 leaf stage. Visual evaluation was initiated after 3 weeks of herbicide application by comparing to non-sprayed wild-type borage. Putative tolerant plants were marked and grown to maturity in the field. The seeds collected from those putative tolerant plants were subjected to herbicide screening again in the growth chamber.

Generation of a Borage Mutant Population by EMS-Induced Mutagenesis.

Approximately 164000 EMS-treated M1 seeds were sown in the field, of which approximately 20,000 germinated, accounting for about 12% of the germination rate (Table 2). As shown in the table, lower concentration of EMS and shorter period of treatment led to higher rate of germination. From M1 borage, a total of 3.5 kg of M2 seeds were harvested, which constituted a mutant population for imidazolinone resistance screening.

TABLE 2
The Germination rate of M1 seeds from EMS-induced mutagenesis

| | Germinated M1 | Sown | Germination |
|---|---|---|---|
| Treatment | seeds | seeds | Rate (%) |
| 0.5% EMS 8 hrs | 3870 | 20718 | 19% |
| 0.5% EMS 16 hrs | 1728 | 20718 | 8% |
| 1.0% EMS 8 hrs | 6104 | 41436 | 15% |
| 1.0% EMS 16 hrs | 3586 | 41436 | 9% |
| 1.5% EMS 8 hrs | 2273 | 20718 | 11% |
| 1.5% EMS 16 hrs | 1602 | 20718 | 8% |

Phenotypic Observation of Mutant Plants.

Phenotypic survey of M1 plants in the field observed many unusual morphological changes. For instance, a normal borage flower has 5 petals, while the abnormal number of petals such as 4 or 6 in flowers was seen in the mutant plants. In addition, dwarf and delayed growth and development plants were frequently observed.

Screening of the Mutant Population for Imidazolinone Resistance.

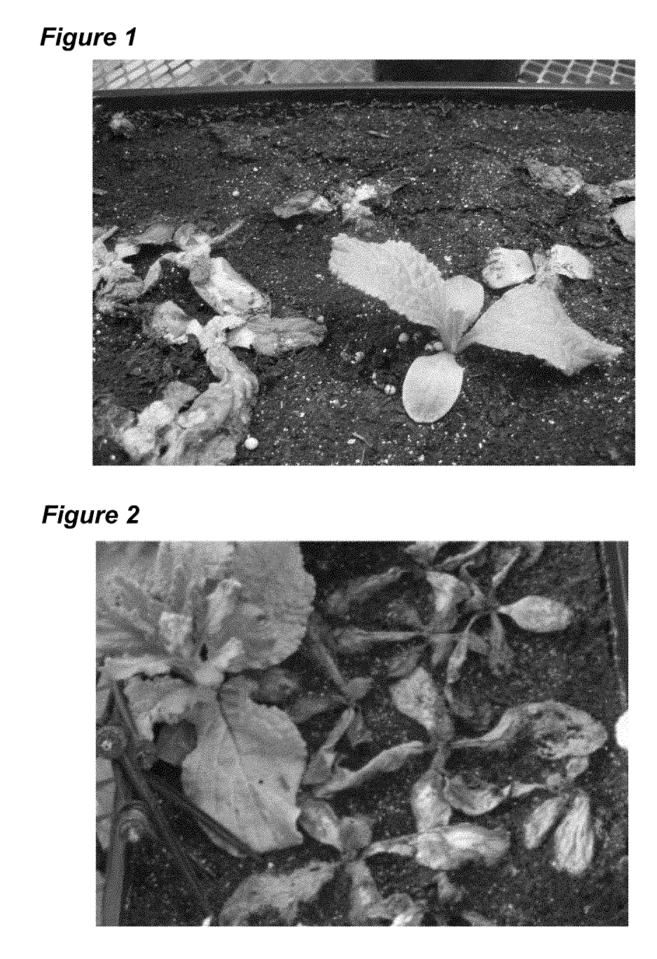

Imidazolinone resistance screening was carried out in a specialized spraying chamber equipped with a moving nozzle herbicide sprayer. About 2X of agronomically recommended dosage of “Solo” herbicide was applied to M2 borage plants. The screening resulted in identification of the first tolerant plants (FIG. 1) from offspring of the M1 seeds that were treated with 1.5% EMS for 16 hours. This plant was then self-pollinated and kept growing to maturity. A total of 271 M3 seeds was harvested from the tolerant plant, and subjected to herbicide screening again. Altogether 225 of 271 M3 seeds were germinated. After herbicide screening by 2X of “Solo” herbicide, 169 plants survived and 56 were killed by the herbicide. The ratio of the imidazolinone-tolerant and imidazolinone-susceptible of the M3 borage plants was 3:1 [X2 (1, N=225)=0.001, p<0.01]. From those surviving plants, 9 of them were transplanted and pollinated by hand to produce M4 seeds. Using the similar screening procedure, the second imidazolinone tolerant borage plant (FIG. 2) was identified in the field.

In summary, three different EMS concentrations from 0.5% to 1.5% and two different time lengths were used for mutagenesis. Among 6 groups of treated M1 seeds, the germination rate ranged from 8% to 19%, which was significantly lower than the normal germination rate (approximately 40-60%). For 8 hour treatments, seeds exposed to higher concentration of EMS had a lower germination rate, while for 16 hour treatments, germination rates were similar at 8% to 9%, despite of different EMS treatment concentrations. Lower rate of germination was observed to associate with longer time of EMS exposure in the study. In addition, pronounced sterility was observed in the group of M1 seeds soaked in 1.5% EMS for 16 hours. The amount of M2 seeds harvested from the group was extremely small, only 65 g in 3.5 Kg of M2 seeds sown. However, it is noted that the first imidazolinone tolerant borage plant was identified from this group of mutant seeds. All M1 and selected M2 tolerant plants did not show any change of flower color; however in selected M3 plants, 5 out of 9 tolerant plants produced white flowers instead of blue ones. White flowers are occasionally observed from natural mutation in the field; however, the blue flower color is genetically dominant over the white flower color. EMS-induced mutations could occur randomly at various locations throughout the genome. Thus, mutations can be found not only in the AHAS genes responsible for imidazolinone tolerance, but also on the locus involved in the biosynthesis of flower pigments. As a result, EMS mutagenesis can generate loss-of and gain-of-function mutants at the same time in a single plant. Identified imidazolinone tolerant borage, thereby, may lose certain good traits after mutagenesis. Further breeding is thus required and highly necessary to integrate imidazolinone resistance trait into a commercial borage line.

EMS mutagenesis can introduce a single nucleotide substitution of one AHAS allele in M1 plants. This means that M2 would be a segregation population on the gene. As AHAS mutation is commonly dominant or semi-dominant, AHAS heterozygous mutant in the population can show the tolerant phenotype to imidazolinones. Homozygous imidazolinone-resistant plants may appear more tolerant than heterozygous individuals, but it is still difficult to distinguish them by visual screening inspection. Therefore, a genotyping marker is needed to identify homogeneity of the mutant allele.

### Example 2

### Cloning of Borage AHAS Genes and Identification of the Point Mutation Responsible for Imidazolinone Resistance

The acetohydroxyacid synthase (AHAS) (EC 4.1.3.18), also known as acetolactate synthase (ALS), catalyzes the first reaction in the pathway for synthesis of branched chain amino acids leucine, isoleucine and valine in plants and microorganisms. AHAS plays a critical role to ensure a balance supply of the amino acids as well as producing intermediates to interact with other cellular metabolic pathways. In plants, the gene encoding AHAS enzyme, AHAS, is generally expressed in a constitutive manner, but expression level may vary between tissues and developmental stages. The identity of AHAS protein sequences among different species ranges from 17% to 90%, and certain key residues of AHAS enzymes are absolutely conserved across species. Imidazolinone herbicides control weeds by inhibiting activity of the native AHAS enzyme, thus natural mutation or chemical induced mutations in AHAS gene could result in the enzyme with less or no sensitivity to imidazolinone herbicides. In reference to Arabidopsis thaliana, five commonly occurring mutations in an AHAS gene for the catalytic subunit at codon locations of Ala122, Pro197, Ala205, Trp574 and Ser653 contribute to tolerance to AHAS inhibitors. Efforts were undertaken to clone AHAS genes from wild-type and mutant borage plants to identify the mutation responsible for the herbicide resistant phenotype.

AHAS protein sequences of Arabidopsis and sunflower were used as queries to BLAST search a partial genome database of borage resulting in identification of many short fragments of DNA sequences that were homologous to the AHAS sequences. Based on the fragment sequences, several sets of primers were designed to retrieve missing ends of borage AHAS genes by RACE-PCR. After retrieving the missing ends, specific 5′ and 3′ end primers were designed to obtain full length AHAS genes. By this process, two homologous AHAS genes, AHAS1 and AHAS2, were cloned from wild-type and imidazolinone-resistant borage plants, respectively. Comparison of these sequences revealed point mutations in two AHAS genes responsible for imidazolinone resistance.

RNA Isolation, RACE-Ready cDNA Synthesis.

Total RNA was extracted from borage leaves. About 0.5-1.0 g leaf tissue was pulverized in liquid nitrogen to fine powder using pestle and mortar. Total RNA was isolated using 1 mL Trizol reagent (Invitrogen Corp) per 50-100 mg of tissue sample according to the manufacturer's recommendations. RNA was quantified by absorbance at 260 nm and 280 nm using a NanoDrop spectrophotometer (NanoDrop Technologies). RACE-Ready cDNAs including both 5′-RACE-Ready cDNA and 3′-RACE-Ready cDNA were synthesized according to SMARTer RACE cDNA amplification manual (Clontech Laboratories). A 3.75 μL mixture of the RNA and 5′-cDNA synthesis primer A was incubated at 72° C. for 3 minutes, then cooled to 42° C. for 2 min. After cooling, the mixture was briefly centrifuged for 10 seconds at 14,000 g, and then 1.0 μL of the SMARTer IIA oligo were added to 5′-RACE-Ready cDNA synthesis reaction. A 4.0 μL buffer mix including 2.0 μL of 5X first strand buffer, 1.0 μL of 20 mM DTT and 1.0 μL of 10 mM dNTPs was combined with 0.25 μL of 40 U/μL RNase inhibitor and 1.0 μL of 100U SMARTSribe reverse transcriptase to form the master mix. The mixtures were incubated in a hot-lid thermal cycler at 42° C. for 90 min, and then heat at 70° C. for 10 minutes. The RACE-Ready cDNA products were diluted with Tris-EDTA buffer and stored at −20° C. Similarly, 3′-RACE-Ready cDNA was synthesized by the same procedure.

Cloning of the Borage AHAS1 Gene.

The sunflower AHAS gene was used as a query to blast search against the database of borage partial genomic sequences by CLC workbench software (CLC Bio). Primers were designed upon the borage DNA fragments that have highest homology with the query sequence. Three reverse primers (AHAS1-5R-R1, AHAS1-5R-R2 and AHAS1-FLR, Table 3) and one forward primer (AHAS1-3RF, Table 3) were designed. 5′ prime end RACE-PCR reaction was carried out in 25 μL reaction mixture, containing 3.35 μL molecular biology grade water, 12.5 μL 2X buffer, 2.5 μL of 2 mM dNTPs (Novagen, EMD Chemicals), 2.5 μL Universal Primer A Mix (UPM, ClonTech Laboratories), 1.25 μL primer AHAS1-5R-R1 or AHAS1-5R-R2 respectively, 2.5 μL 5′ end cDNA and 0.4 μL KOD Xtreme Hot Start DNA polymerase (Novagen, EMD Chemicals). The PCR profile was as follows: initial denaturation, 94° C. for 3 minutes; 3 cycles X (94° C. for 30 s, 72° C. for 80 s); 5 cycles X (94° C. for 30 s, 68° C. for 30 s, 72° C. for 80 s); 25 cycles X (94° C. for 30 s, 63.5° C. for 30 s, 72° C. for 80 s); and final extension at 72° C. for 10 minutes.

TABLE 3
Primers for retrieving borage
AHAS1 & AHAS2 genes

| | | SEQ |
|---|---|---|
| Primer | Sequence | ID NO: |
| AHAS1 5RR1 | CCAATCATCCTACGAGGTACTTGTCCAG | 1 |
| AHAS1 5RR2 | GCAAAAACTCCTCCCTGTTCATGCCTAG | 2 |
| AHAS1 3RF | ACGTGCTTCCTAGGCATGAACAGGGA | 3 |
| AHAS1 FLR | ACACGGTGAACTCGTCTAACCTTGAGGA | 4 |
| AHAS1 FLF | GAAGCCATGGGGATCTCCTCACATTTCACAACC | 5 |
| AHAS2 5RR1 | TTGTCCAACACCGGTACTTATGATTGCAT | 6 |
| AHAS2 5RR2 | TAGCATCTCCAAACGTTTTAAATGTCAACG | 7 |
| AHAS2 3RF1 | TCCTCGTAGATGATTGGTACTGATGCG | 8 |
| AHAS2 3RF2 | GCCTGGCCCGGTTTTGATTGACGT | 9 |
| AHAS2 FLR | TGAAATACAACGCAAGTCAAACTCTAC | 10 |
| AHAS2 FLF | TCTCCACCACTCTCTTCACCGTC | 11 |

The amplification products were resolved on 1% agarose gel. A lkb plus DNA ladder was used as a size marker (New England BioLabs). Bands of expected sizes were excised from the gel and DNA was eluted from the bands using the EZ-10 spin column gel extraction kit following the manufacturer's protocol (Bio Basic Inc.). The eluted DNA was verified by nested RACE-PCR using another reverse primer AHAS1-5R-R2. The nested RACE-PCR reaction contained 2.5 μL Nested Universal Primer A (NUP, ClonTech Laboratories) and 1.25 μL primer AHAS1-5R-R2. After amplification, the products were separated by agarose electrophoresis and one band with correct size was excised from the gel. The DNA was eluted from the band.

To clone the fragment, the 5′-RACE DNA fragment was first extended with poly-As using Taq polymerase and then 3 μL PCR product was mixed with 5 μL 2X rapid ligation buffer, 1 μL pGEM-T vector and 1 μL T4 DNA ligase (Promega Corp). The mixture was incubated at 4° C. overnight for ligation. A 2 μL aliquot of the ligation was transformed into 35 μL of E. coli Top Ten cells by electrophoresis. After incubation at 37° C. for 1 hour, competent cells were spread onto prepared selecting plates. After incubation at 37° C. for 16 to 24 hours, plates were examined for white colonies which are indicative of transformants. White colonies were picked and incubated individually at 37° C. for 16 to 24 hours. Concurrently, colony PCR containing 18.3 μL molecular biology grade water, 2.5 μL 10X buffer, 2.5 μL MgSO₄, 0.5 μL of 10 mM dNTPs, 0.5 μL NUP, 0.5 μL primer AHAS1-5R-R2 respectively, 0.2 μL Taq polymerase and a dip of colonies as template, were performed to verify the transformants. The PCR profile was as follows: initial denaturation, 95° C. for 2 minutes; 30 cycles X (95° C. for 30 s, 62° C. for 30 s, 72° C. for 2 min); and final extension at 72° C. for 10 minutes. Plasmid DNAs in positive transformants was isolated and purified using the EZ-10 spin column plasmid DNA kit following the manufacturer's protocol (Bio Basic Inc.). The DNA was quantified by absorbance at 260 nm and 280 nm using a NanoDrop spectrophotometer before sequencing. PCR amplification of 3′ ends was less complicated because 3′ end could be obtained from the borage genomic database. Simple PCR was carried out in 50 μL reaction mixture, containing 33.5 μL of molecular biology grade water, 5 μL 10X Pfu buffer (Bio Basic Inc.), 1 μL of 10 mM dNTPs, 2.5 μL AHAS1-3RF, 2.5 μL AHAS1-FLR, 5 μL cNDA and 0.5 μL Pfu DNA polymerase (Bio Basic Inc.). PCR profile was as follows: initial denaturation, 98° C. for 2 minutes; 35 cycles X (98° C. for 30 s, 68° C. for 30 s, 72° C. for 2 minutes); final extension at 72° C. for 10 minutes.

The 5′ and 3′ ends of AHAS1 gene were assembled using Vector NTI software (Invitrogen). According to the putative full length of AHAS1, a forward primer (AHAS1-FLF) from 5′ end was designed. Full-length gene was amplified by Pfu PCR reaction in 50 μL reaction mixture, containing 33.5 μL molecular biology grade water, 5 μL 10X phusion buffer, 1 μL of 10 mM dNTPs, 2.5 μL AHAS1-FLF, 2.5 μL AHAS1-FLR, 5 μL cNDA and 0.5 μL Pfu DNA polymerase. PCR profile was as follows: initial denaturation at 98° C. for 2 minutes; 35 cycles X (98° C. for 30 s, 65° C. for 30 s, 72° C. for 2.5 minutes); final extension at 72° C. for 10 minutes. A full length AHAS1 gene was obtained following the same procedure described above including gel DNA extraction, poly-A overhang, ligation to pGEM-T vector, transformation of the vector to E. coli, colony selection and plasmid DNA extraction and sequencing. Due to the length of the AHAS1 gene, an extra sequencing primer, AHAS1 3RF, was used to obtain the middle part of the sequence.

Cloning of Borage AHAS2 Gene.

Borage AHAS1 gene was used as a query to BLAST search against the database of borage genomic sequences by CLC workbench software (CLC Bio). Homologous partial sequences were assembled and compared with AHAS1 gene sequence, which indicated the presence of a second AHAS gene in borage. Two forward primers (AHAS2-3RF1 & AHAS2-3RF2, Table 3) and two reverse primers (AHAS2-5RR1 & AHAS2-5RR2, Table 3) were designed for retrieving the AHAS2 gene. Five prime end and 3′ end RACE-PCR reactions were carried out in 25 μL reaction mixture, containing 3.35 μL molecular biology grade water, 12.5 μL 2X buffer (Novagen, EMD Chemicals), 2.5 μL of 2 mM dNTPs, 2.5 μL Universal Primer A Mix (UPM, ClonTech Laboratories), 1.25 μL primer AHAS2-5RR1 and AHAS2-5RR2 each (AHAS2-3RF1 and AHAS2-3RF2 for 3′ end RACE-PCR), 2.5 μL 5′ end cNDA (3′ end cNDA for 3′ end RACE-PCR) and 0.4 μL KOD Xtreme Hot Start DNA Polymerase (Novagen, EMD Chemicals). PCR profile was as follows: initial denaturation: 94° C. for 3 minutes; 3 cycles X (94° C. for 30 s, 72° C. for 80 s); 5 cycles X (94° C. for 30 s, 68° C. for 30 s, 72° C. for 80 s); 35 cycles X (94° C. for 30 s, 63° C. for 30 s, 72° C. for 80 s); the final extension at 72° C. for 10 minutes. The rest of assembly, amplification and cloning of AHAS2 genes followed the same procedure as described in the section of cloning AHAS1 gene.

Identification of Point Mutations in AHAS Genes Responsible for Imidazolinone Resistance.

Using the same primer sets, AHAS1 and AHAS2 from imidazolinone resistant borage were amplified, cloned and sequenced following the exactly same procedure as described above. By comparing AHAS genes between wild-type and resistant borage plants using Vector NTI software (Invitrogen), point mutations responsible for imidazolinone resistance were finally identified for both imidazolinone resistant lines.

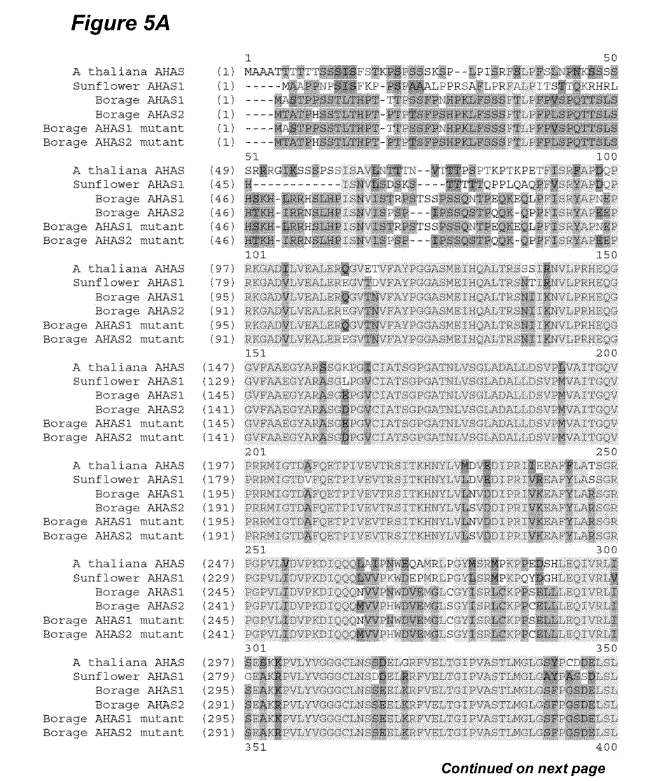

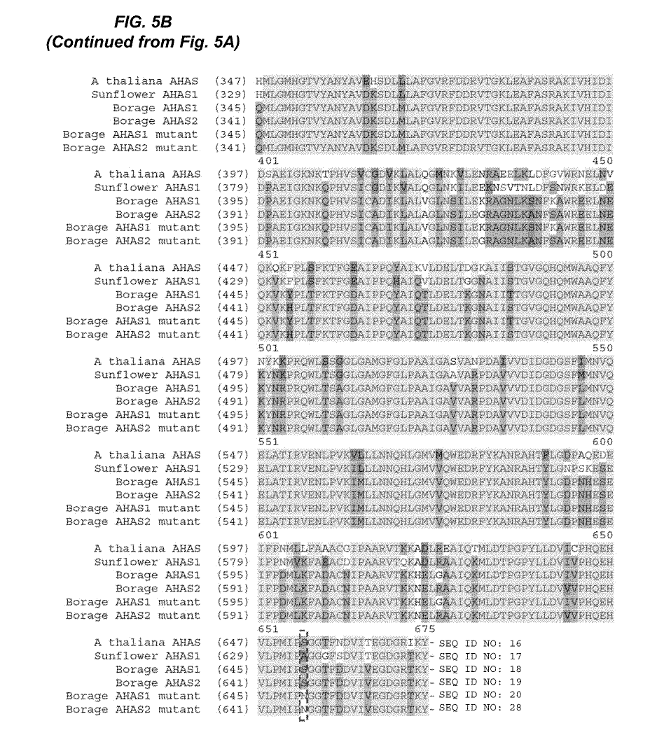

In summary, using Arabidopsis and sunflower AHAS sequences as queries to search a partial genomic sequence database, two homologous AHAS genes were identified in borage. However, the two borage sequences were not in full-length. To obtain the missing 5′ and 3′ ends of the AHAS genes, RACE-PCR approach was used to retrieve the sequence information of the missing ends. Analysis of the assembled full-length AHAS1 and AHAS2 genes (FIGS. 5A and 5B) indicated that the open reading frame (ORF) of AHAS1 was 2007 bp in length encoding a protein of 669 amino acids, and the ORF of AHAS2 was 1995 bp long encoding a polypeptide of 665 amino acids.

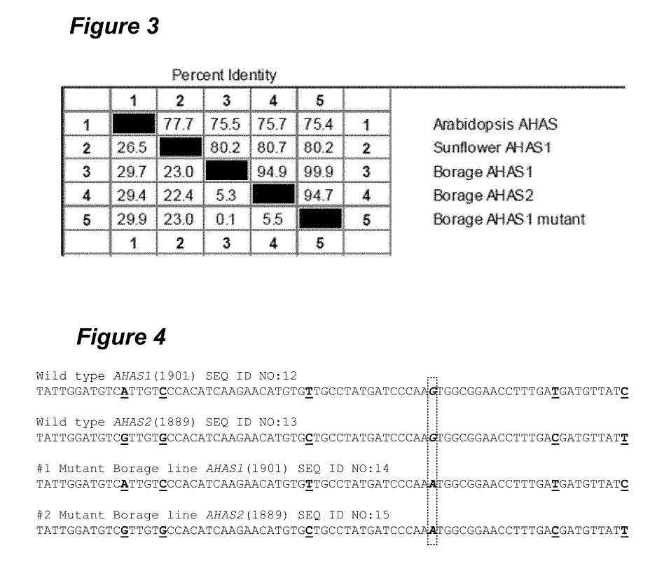

Protein sequence comparison of borage and other plants AHASs showed that borage AHAS1 and AHAS2 were 95% identical at the amino acid level and both shared approximately 75% of amino acid identity with Arabidopsis AHAS protein and 80% with sunflower AHAS1 protein sequence (FIG. 3). The comparison of AHAS genes isolated from the wild-type and two imidazolinone resistant lines revealed that one single nucleotide substitution (from G to A) occurred in AHAS1 gene at 1953 bp (FIG. 4) in the first resistant line, which resulted in an amino acid change at position 651 from serine (S) in the wild type to asparagine (N) in the mutant (FIG. 5). Interestingly enough, the second resistant line has the same single nucleotide substitution in AHAS2, but not AHAS1, at 194 lbp (FIG. 4) resulting in the same amino acid change at position 647 (FIG. 5).

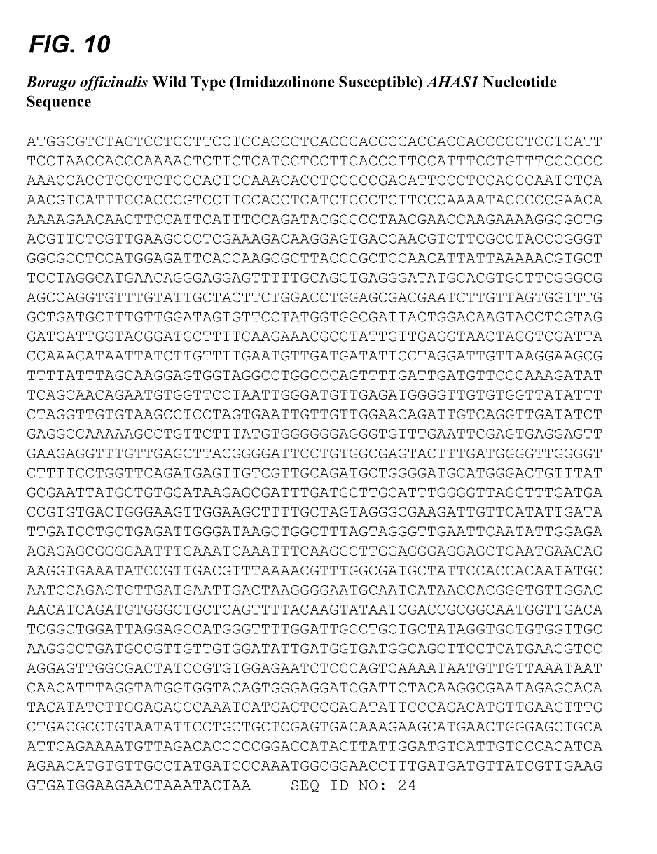

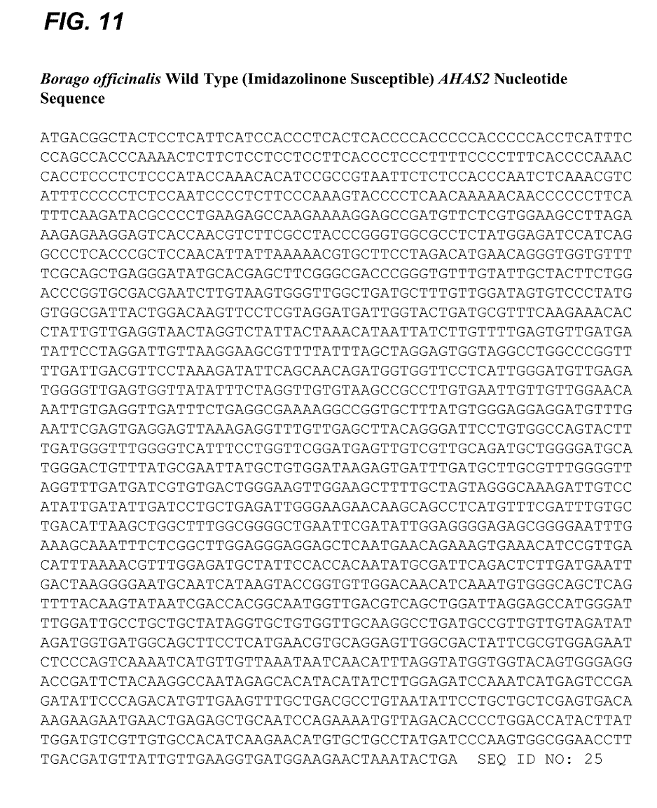

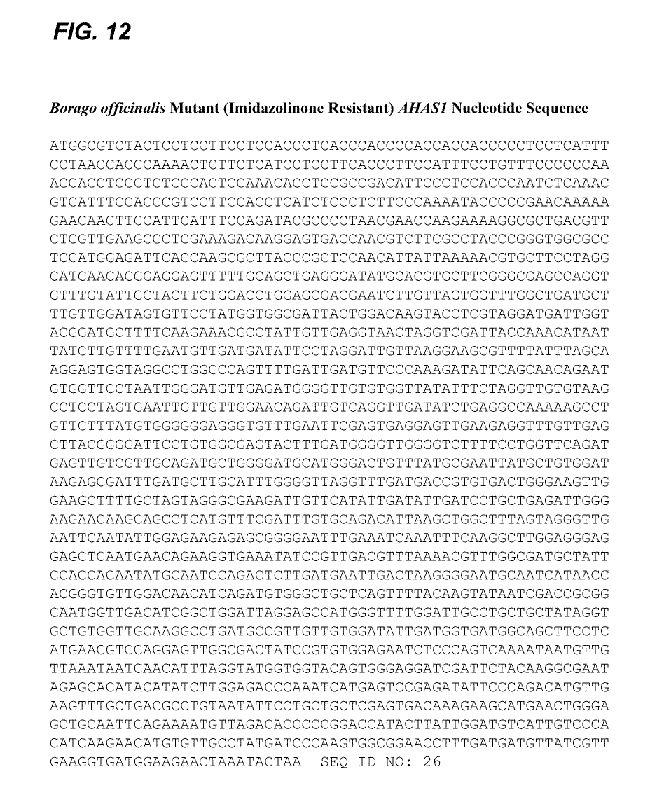

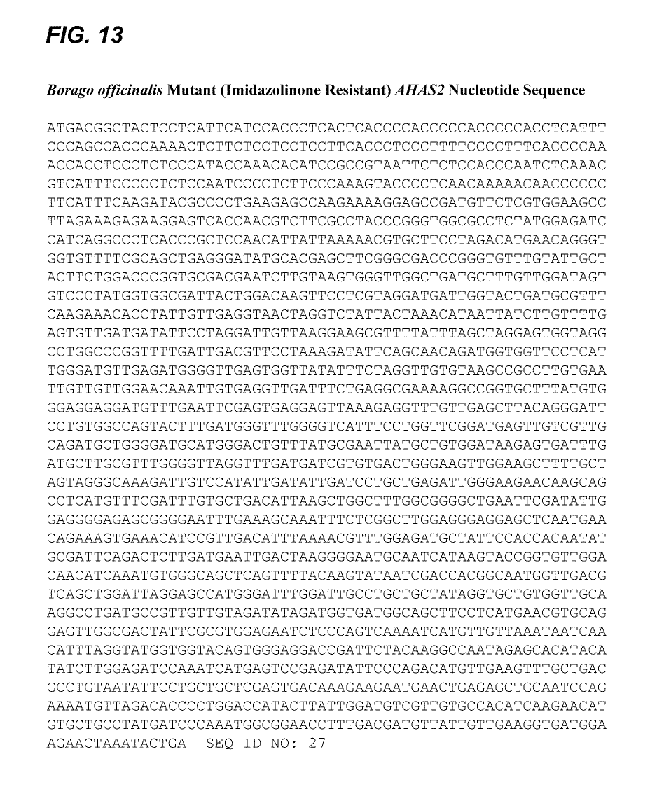

In summary, two AHAS genes were isolated by chemical induced mutagenesis in borage. The genes showed very high homology with each other, up to 95% identity at the amino acid level. FIG. 10 shows the deduced Borago officinalis Wild Type (Imidazolinone Susceptible) AHAS2 Nucleotide Sequence. FIG. 11 shows the Borago officinalis Wild Type (Imidazolinone Susceptible) AHAS2 Nucleotide Sequence. FIG. 12 shows the Borago officinalis Mutant (Imidazolinone Resistant) AHAS1 Nucleotide Sequence. FIG. 13 shows the Borago officinalis Mutant (Imidazolinone Resistant) AHAS2 Nucleotide Sequence.

Both AHAS protein sequences also share greater than 75% identity with Arabidopsis AHAS and sunflower AHAS. AHAS genes have been identified and sequenced in a variety of plants, fungi, algae and bacteria. The similarity of AHAS protein sequences among different species ranges from 17% to 90%. Many residues of AHAS enzymes are conserved across species. In most plant species, at least one AHAS gene is expressed in a constitutive manner, as AHAS is known as housekeeping gene. Some plants, such as N. tabacum, B. napus and G. hirsutum, contain more than one AHAS genes. The presence of multiple AHAS genes may be derived from a polyploidy process by the combination of genomes of their diploid progenitors. In some plants, there are two housekeeping AHAS genes expressed at about same level. In B. napus and G. hirsutum, there is another AHAS gene expressed in tissue specific manner. Interestingly, B. napus also contains a fourth AHAS gene which is considered as a pseudogene and is not expressed. The present isolation and cloning effort revealed that borage has at least two AHAS genes that are constitutively expressed as both cDNAs were retrieved from the leaf tissue.

Sequence comparison identified point mutations in the coding region of AHAS1 and AHAS2 respectively in two different imidazolinone resistant borage plants. The point mutation results in an amino acid change from serine to asparagine in the AHAS proteins (FIG. 5). Previous studies showed the most common mutations for herbicide resistance are at residues A122, P197, A205, W574 and S653 referring to Arabidopsis AHAS protein sequence. Duggleby R G and Pang S S. (2000), supra. According to the early research, the amino acid substitution resides in the γ-domain at the C-terminal end of the catalytic subunit of AHAS enzymes. The catalytic subunit aggregates to form a tetramer complex with another tetramer of four regulatory subunits to constitute the AHAS apoenzyme. The serine residue in the position of AHAS enzymes is relatively conserved across species, although there is an alanine in the cocklebur enzyme, a glycine in the yeast enzyme and in E. coli AHAS III, and a proline in E. coli AHAS I and II. See Bernasconi P, et al. A naturally occurring point mutation confers broad range tolerance to herbicides that target acetolactate synthase. Journal of Biological Chemistry 270 (1995) 17381-17385; Sathasvian K, et al. Molecular basis of imidazolinone herbicide resistance in Arabidopsis thaliana var columbia. Plant Physiology 97 (1991) 1044-1050. In addition to resistance to imidazolinones, the mutation at this site is characterized by cross-resistance to pyrimidyl oxybenzoates, but not to sulfonylureas and triazolopyrimidines. See id. Sathasivan et al. Using in vitro mutagenesis, the serine residue was mutated to different amino acids such as alanine, threonine and phenylalanine. The alanine substitution is sensitive to sulfonylureas and imidazolinones, while S653T, S653N and S653F mutations result in enzymes with 10 fold or more resistance to imidazolinones. See Duggleby and Pang, 2000, supra.

### Example 3

### Development of KASP SNP Marker Linked to Imidazolinone Resistance Gene (AHAS1 Mutation) in Borage

The herbicide resistant mutation is generally dominant; therefore genotypes of homozygous and heterozygous tolerant plants are difficult to distinguish from their phenotypes. However, since imizadolinone resistance of borage is induced by a single nucleotide substitution, the SNP can be detected by KASP technology and used as a marker to distinguish the genotypes. The KASP genotyping system is an accurate and cost-effective fluorescence-based technology developed by KBioscience for high-throughput SNPs genotyping.

The technology is based on allele-specific oligo extension and fluorescence resonance energy transfer (FRET) for signal generation. Two allele-specific primers and one allele common primer are included in a KASP genotyping assay. Each allele-specific primer must be ended with a specific nucleotide of the SNP and attached with unique unlabeled tail sequences at the 5′ end. The mixture of FAM and HEX specific FRET cassettes in the master mix will bind the unique target tail sequences to produce fluorescence with either only one or a mixed type of the signal (LGC Genomics, 2014). According to the signal generated, sample materials can be assigned to homozygous and heterozygous genotypes. KASP technology was used to genotype the herbicide resistant borage plants and differentiate between homozygous herbicide-resistant, homozygous herbicide-susceptible and heterozygous herbicide-resistant borage genotypes.

Based on the single nucleotide substitution of the AHAS1 gene in the herbicide resistant borage, a set of primers was designed to develop the KASP SNP marker. The result showed that the marker could readily distinguish wild type, heterozygous and homozygous plants. This robust and user-friendly KASP SNP marker was determined to be useful for routine marker-assisted selection of imidazolinone resistant borage.

Forty M3 borage plants from a single AHAS1 mutant line were randomly selected and numbered. Their leaf tissues were collected at the 2-4 leaf stage. Leaf samples were snap-frozen in liquid nitrogen and stored at −80° C. for genomic DNA extraction. Forty plants were kept for imidazolinone screening to validate the result of KASP genotyping. Genomic DNA was extracted by adapting the CTAB method (Dietrich C R, et al. Mu transposon insertions are targeted to the 5′ UTR of the maize g18 gene. Genetics 160 (2002) 697-716). The DNA obtained was diluted to 5 ng/μL. DNA samples were stored at −20° C. for further use. A set of primers (Table 4) was designed and synthesized for KASP genotyping following the manual from LGC genomics. Each set of primers consists of two gene-specific primers and one common primer. Gene-specific primers contain a unique unlabeled tail sequence at the 5′ end. Gene-specific primers have to end with the SNP at the 3′ end. In this case, two specific primers are reverse primers and the common primer is forward.

TABLE 4
Primers for KASP genotyping

| | | SEQ |
|---|---|---|
| Primer | Sequence | ID NO: |
| Reverse Allele- | GAAGGTCGGAGTCAA | 21 |
| Specific Primer 1 | CGGATTGATAACATC | |
| | ATCAAAGGTTCCGCC | |
| | AT | |
| Reverse Allele- | GAAGGTGACCAAGTT | 22 |
| Specific Primer 2 | CATGCTATAACATCA | |
| | TCAAAGGTTCCGCCA | |
| | C | |
| Forward Common | CGGACCATACTTATT | 23 |
| Primer | GGATGTCATTGTC | |

The Bold highlighted sequence in primer 1 is unlabeled oligo sequence and primer 1 ends with mutant nucleotide “T”; The Bold highlighted sequence in primer 2 is unlabeled oligo sequence and primer 2 ends with original nucleotide “C”).

KASP genotyping assay was performed on StepOne Real-time PCR system (Applied Bioisystems). On a 96-well plate, each well contained about 10 μL of reaction mixture, including 5 μL of DNA sample, 5 μL of 2X master mix (LGC genomics), 0.14 μL of primer mix. Four homozygous susceptible controls and two no-template controls were also included. KASP assay thermal cycling program was as follows: pre-read at 25° C. for 30 s; holding at 95° C. for 15 s; 10 cycles X (95° C. for 20 s, 61° C. for 60 s); 30 cycles X (95° C. for 20 s, 55° C. for 60 s); post-read at 25° C. for 30 s.

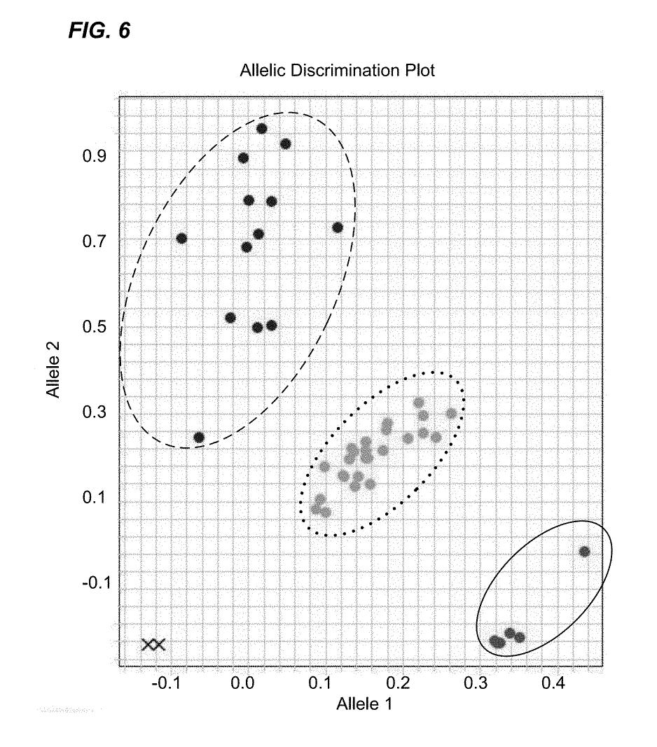

The individual genotype of a segregating population of forty M3 borage samples was determined by KASP genotyping PCR. The allelic discrimination plot based on the PCR result was shown in FIG. 6 and the segregation result of 40 borage samples based on KASP genotyping was summarized in Table 5. As shown in FIG. 6, the plant genotypes could be divided into three groups. The homozygous herbicide resistant plants represented by homozygous FAM (fluorescein amidite) allele marked by red were clustered in the lower right corner of the plot (solid oval), the homozygous susceptible plants represented by the homozygous HEX (5-hexadecanoyl fluorescein) allele marked by blue were clustered in the upper left corner of the plot (dashed oval), while the heterozygous plants marked by green are clustered in the central region of the plot (dotted oval). It was noted that the KASP genotypes of individuals was consistent with their herbicide spraying phenotype and overall ratio of herbicide tolerant plants to susceptible plants was 31:9, equivalent to the theoretical ratio 3:1 [X₂(1, N=40)=0.133,p<0.01] (Table 5).

TABLE 5
Segregation of M3 borage plants based on the KASP
genotyping result. The homozygous resistant, RR;
the homozygous susceptible, rr; the heterozygous, Rr.
Segregation of M3 Borage Plants

| Resistant (RR) | Susceptible (rr) | Heterozygous (Rr) |
|---|---|---|
| 6 | 9 | 25 |

In summary, a KASP assay was developed and successfully used to identify and differentiate the homozygous resistant, homozygous susceptible and heterozygous plants in the segregation population. The result was confirmed by phenotyping of the herbicide resistance. The herbicide screening showed among the 40 plants, 9 of them were susceptible and 31 were tolerant, which was consistent with those identified by the KASP genotyping result. As described previously herein, the M3 borage population was expected to segregate into imidazolinone resistant and susceptible groups by 3:1 ratio. The number of the resistant to the susceptible at 31:9 from the KASP genotyping (Table 5) is close to the expected ratio. In addition, imidazolinone susceptible controls and no-template controls are readily distinguished in the assay, indicating that the sensitivity of KASP assay is excellent. Therefore, the KASP assay is being used to select imidazolinone resistant traits for rapid genotyping and seed increase in our breeding program. After the KASP genotyping and herbicide-screening phenotyping validation, approximately 20 homozygous resistant plants of the M4 generation were determined and selected for the breeding.

### Example 4

### In Vitro AHAS Activity Assays of the Resistant and Susceptible Borage Plants

The herbicide imazamox is an inhibitor of the AHAS enzyme. Homozygous herbicide resistant borage line (AHAS1 mutant) should retain significantly higher AHAS activity than the herbicide-susceptible borage (wild-type) when assayed in presence of the inhibitor. It has been shown from the above Examples that the AHAS1 mutant borage line can tolerate two times the recommended dosage of “Solo” herbicide and the tolerance is caused by the single nucleotide mutation of the AHAS1 gene. As such, the AHAS activity level of the mutant plants would provide further evidence to support the conclusion. AHAS enzyme activity is generally measured using a discontinuous colorimetric assay based on the method developed by Singh et al. (Singh B K, et al. Assay of acetohydroxyacid synthase. Analytical Biochemistry 171 (1988) 173-179). In this method, the crude protein is incubated with the substrate for a fixed time to generate intermediate acetolactate that is then converted to acetoin under decarboxylation. Finally, the reaction of acetoin with creatine and α-naphthol forms a pink-colored product which can be measured at 520 nm wavelength in a spectrometer. As a result, the AHAS activity level can be estimated based on the color density. See Duggleby R G and Pang S S. (2000) supra.

To determine AHAS enzyme activity with the borage mutation, crude proteins from leaf tissues of both susceptible (wild-type) and resistant (AHAS1 mutant) plants were extracted and used as enzyme sources for the AHAS activity assay. The protein extract containing the AHAS enzymes was incubated with the substrate and cofactors in a buffer with or without imazamox, the herbicide active ingredient. The catalytic reaction transformed the substrate to acetolactate that was then further converted to acetoin. The detection of acetoin via the formation of a creatine and naphthol complex was used to determine the AHAS activity of susceptible and resistant plants.

Preparation of Enzyme Sources.

An in vitro assay of AHAS activity for each imazamox concentration (0, 1, 5, 25, 125, 625 μM) was performed with three biological samples per genotype, and each biological sample consisted of two technical replicates. At the 4-6 leaf stage, the leaf material (about 3-4 g) was harvested and snap-frozen in liquid nitrogen and stored at −80° C. The in vitro assay was conducted as follows. About 1 g of the frozen material was ground to a fine powder with a mortar and pestle in liquid nitrogen and homogenized in 4 volumes of cold extraction buffer containing 0.1 M K₂HPO₄ (pH 7.5), 10 mM sodium pyruvate, 0.5 mM MgCl₂, 0.5 mM thiamine pyrophosphate (TPP), 10 uM flavin adenine dinucleotide (FAD), 4 mM DTT, 1 mM phenylmethylsulphonyl fluoride (PMSF), 10% v/v glycerol, and 4% soluble PVP. The homogenate was filtered through two layers of miracloth and the filtrate was centrifuged at 30,000 g for 20 minutes at 4° C. The supernatant was firstly brought to 30% saturation by drop-wise addition of solid (NH₄)₂SO₄ to allow unknown “gums” to form and to be removed; the supernatant was then brought to 50% saturation with (NH₄)₂SO₄. The solution was allowed to stand on ice for 10 min with occasional stirring such that any additional “gums” would be removed or filtered out. The sample solution was then divided into two as technical replicates before going to centrifugation. After centrifugation at 100,000 g for 20 minutes at 4° C., the gummy protein layer was carefully collected as enzyme sources for the activity assay.

Enzyme Incubation and Colorimetric Reaction.

The gummy protein layer was re-dissolved in 1.4 mL incubation buffer containing 50 mM K₂HPO₄ buffer (pH 7.0), 100 mM sodium pyruvate, 10 mM MgCl₂, 1 mM TPP and 1 μM FAD. The amount of protein in each sample was determined immediately by the Bio-Rad protein assay using a dye reagent (#500-0006). A series of concentrations at 0, 1, 5, 25, 125, 625 μM imazamox PESTANAL®, analytical standard (Sigma-Aldrich Co. LLC) were added to 200 μL of the reaction mixture, respectively. The mixture was incubated at 37° C. for 1 hour. To stop the reaction, 32 μL of 1 M H₂SO₄ was added and decarboxylation occurred at 65° C. for 15 minutes. Then the sample was incubated with 34 μL of creatine solution (1% w/v in 2N NaOH) and 68 μL of α-napthol solution (5% w/v in 2N NaOH) at 60° C. for 15 minutes. After cooling for 10 minutes at the room temperature to maximize the color development, the mixture was briefly centrifuged at 13000 g. 200 μL of the reaction solution was transferred to a 96 well microtiter plate for measurement of the absorbance at 520 nm. The background control for non-AHAS activity was determined by adding 32 μL of 5N NaOH to 200 μL of the reaction mixture after 1 hour of incubation. The unit of enzyme activity was defined as micromole of acetoin produced, and specific activity of AHAS enzyme in resistant and susceptible borage was calculated on the basis of micromole acetoin produced per milligram of the protein and per minute of the reaction time. Therefore, in order to quantify enzymatic levels, standard curves of acetoin were generated using a series of acetoin dilutions in the incubation buffer.

In summary, the in vitro AHAS activity assay was based on measurement of acetoin produced by the AHAS enzyme in presence of the substrate. The production of acetoin shown by pink color products was measured by the colorimetric absorbance reflecting the activity of AHAS enzyme. The higher the activity, the stronger the pinkness. Visual inspection of the assay (not shown) showed that the intensity of the pink color of the acetoin complex produced in both the mutant and wildtype was gradually reduced when imazamox concentrations were increased in the assays. However the pinkness of the resistant line remained stronger than that of the susceptible line, especially at high concentrations of imazamox from 25 μM to 625 μM. This result indicated that although increased inhibition of the total AHAS enzyme activity occurred with increased concentration of imazamox in both borage lines, the imidazolinone resistant mutant line was able to retain significantly greater AHAS activity than the susceptible wild type in the range of imazamox concentrations.

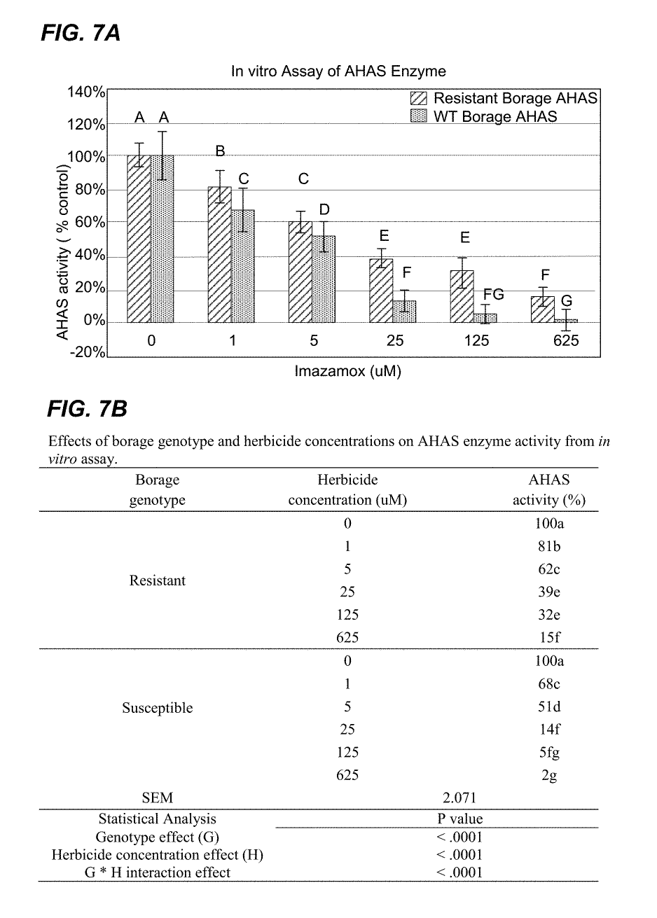

The quantitative result of specific AHAS activity across a range of imazamox concentration between the two genotypes is shown in FIG. 7A, which provide a comparison of specific AHAS activities between the AHAS1 mutant and wild type across different imazamox concentrations. The activity at 0 μM imazamox was as 100%; the same letter means that the activities are not significantly different (P >0.05). As depicted in FIG. 7B, statistical analysis of the data using Factorial Treatment Arrangement on CRD (completely randomized design) indicated that the mutant borage had significantly higher AHAS activity than the wild type borage across all imazamox concentrations, although the specific AHAS activities in both lines were gradually decreased with the imazamox concentrations increased. The activity of the mutant line could retain up to 20% of the total activity at zero μM of imazamox while that of susceptible borage went down to zero at 625 μM of imazamox.

In summary, in the AHAS activity assay, a background control representing acetoin production by non-AHAS activity was included, as a number of acetoin-forming enzymes including pyruvate decarboxylase (PDC) in plant tissues might interfere with the assay. These non-AHAS enzymes catalyze formation of acetoin via non-acidic conversion, which could be estimated using NaOH instead of H₂SO₄ to terminate the reaction. In addition, non-AHAS enzymes such as PDC have been shown to reduce the sensitivity of AHAS enzymes to herbicide or feedback inhibition of branched chain amino acids in maize kernels. The borage AHAS assay in this study showed that acetoin produced by non-AHAS enzymes in leaf tissues accounts for approximately 28% of the total acetoin production (data not shown) and this part of acetoin production was thus excluded from the AHAS activity. As seen in FIG. 7A, in vitro AHAS activity of imidazolinone resistant borage was significantly higher than susceptible borage across all imazamox concentrations tested. The activity of the resistant line could retain up to 20% of the total activity while that of susceptible borage went down to zero at 625 μM imazamox. This result is in agreement with the early research that S653N mutation of an AHAS gene could confer strong tolerance to imidazolinones (Duggleby and Pang, 2000, supra). The serine residue at position 653 located at substrate binding channel is critical for interaction with a substrate, and substitution of the amino acid to asparagine would prevent the herbicide from binding to AHAS, resulting in insensitivity and tolerance of the enzyme to imidazolinones. One of another possible explanation for higher AHAS activity of the resistant line is that the mutant AHAS gene confers increased enzymatic stability with the wild type plant AHAS being more sensitive to the enzyme extraction and purification procedurte. In addition, the higher activity of AHAS in the mutant line may be due to improved ability of cofactor binding or improved stability in the catalytic subunit when interacting with the regulatory subunit of AHAS enzyme.

Although imidazolinone resistant borage showed significantly higher AHAS activity compared to the wild type in the assay, the overall enzyme activity in presence of imazamox was gradually decreased with the concentrations increased. As discussed above, borage has two AHAS genes in the genome that are co-expressed in leaf tissues. If one AHAS gene is mutated in the resistant line (AHAS1), the other (AHAS2) would remain intact. As a result, the total AHAS activity would be reduced even in the AHAS1 mutant line as AHAS2 enzyme is inhibited by the herbicide. However, with the concentration of the imazamox increasing to a very high level, such as 625 μM, the mutant line also becomes less tolerant to the herbicide, resulting in approximately 20% of the total AHAS activity, while the AHAS activity of the susceptible line is completely inhibited by such level of herbicide. There are reports that in vitro AHAS activity is only slightly reduced or unchanged in resistant lines of some dicot plant species. Thus, the resistance difference between in vitro AHAS activity and the whole-plant performance can be related to the number of AHAS genes in a plant species, and the location of a mutation in an AHAS gene as well as the binding affinity of an herbicide and the toxic strength of the herbicide to the target enzyme.

Borage contains a high level of polyphenols in leaf tissues. These polyphenolic compounds may inhibit enzyme activity directly or indirectly by hydrogen bonding with peptide bond oxygens or by covalent modification of amino acid residues. Therefore, in order to remove or inactivate the polyphenols, polyvinylpyrrolidone (PVP) or polyvinylpolypyrolidone (PVPP) and dithiothreitol (DDT) was included in the enzyme extraction buffer to reduce polyphenol interference and maintain a strong reducing environment to counteract the effect of phenol oxidases. Nevertheless, an unknown yellow gummy layer was still observed after precipitation of the enzymatic extract which appeared to be able to bind, adhere or absorb the AHAS enzyme although it did not affect the AHAS activity in a very dramatic way.

### Example 5

### Herbicide Type and Dosage Responses of the AHAS1 Mutant Borage Line

A series of concentrations of a “Solo” herbicide was applied to the borage plants of the mutant line (AHAS1) in a greenhouse to test the resistance level. The result indicated that the mutant borage was tolerant to four times the field applied concentration of the “Solo” herbicide. In addition, different types of the group 2 herbicides were also tested to determine whether the AHAS1 mutation would confer any cross-resistance among the group 2 herbicides. The result showed that the AHAS1 mutation exhibited strong resistance to both imazethapyr and imazamox herbicides as well as some tolerance to flucarbazone herbicide.

The mutant borage plants were selected from tolerance to imazamox, the active ingredient of an imidazolinone herbicide. It was considered possible that it can also be tolerant to other imidazolinone herbicides within the group with the dosage response. The AHAS1 mutant borage line with the single amino acid substitution (S653N) was obtained by screening an EMS mutagenized population using two times the recommended dosage of “Solo” herbicide. According to Duggleby and Pang (2000) supra, the mutation S653N of AHAS gene results in Arabidopsis thaliana with resistance of 100 fold or more to imidazolinones. In addition, other studies showed that the S653 mutation in other species confers tolerance only to imidazolinones, but not to other chemical families in group 2 herbicides. The present study determined the type and level of herbicide resistance conferred by the AHAS1 mutant line.

Herbicide Dosage Response Test.

Herbicide response tests were carried out in a greenhouse in the Innovation Place (Saskatoon). M4 homozygous imidazolinone-resistant borage (AHAS1 mutant line) were planted at 1-2 cm in 25×50 cm flats containing commercial potting mix (Sunshine Mix 3; Sun Gro.) in the growth chamber under a 16 hour light (22° C.) and 8 hour dark (16° C.) cycle. Each flat contained 36 seeds. A group 2 herbicide, “Solo”, was applied over foliage when most plants were at two-leaf stage in an herbicide chamber. The spray solutions included 2X (84 g ai/ha imazamox), 4X, 8X, 16X, 32X, 64X, 128X and 256X of “Solo” with adjuvant Merge at 0.5% (v/v) of the solution volume. A moving nozzle cabinet sprayer with a flat-fan nozzle tip was calibrated to deliver 102 L/ha of the spray solution in a single pass. Sprayed M4 plants were visually evaluated at 21 days after imazamox application by comparing with untreated controls.

Herbicide Type Response Test.

By following a similar procedure above, M4 homozygous imidazolinone-resistant borage (AHAS1 mutant) was tested with 8 types of group 2 herbicides including Solo (84 g ai/ha imazamox, BASF), Muster (45 g ai/ha ethametsulfuron-methyl, DuPont), Pursuit (102 g ai/ha imazethapyr, BASF), Everest 2.0 (116 g ai/ha flucarbazone, Arysta LifeScience), PrePass XC (20 g ai/ha florasulam, Dow AgroScience), Pinnacle SG (11 g ai/ha thifensulfuron, DuPont), Express SG (32 g ai/ha tribenuron methyl, DuPont) and Accent (51 g ai/ha nicosulfuron, DuPont) at 2X of the recommended rate. Adjuvant reagent for each herbicide was added into spray solution accordingly. Sprayed M4 borage plants were visually evaluated at 21 days after spraying by comparing with wild-type controls.

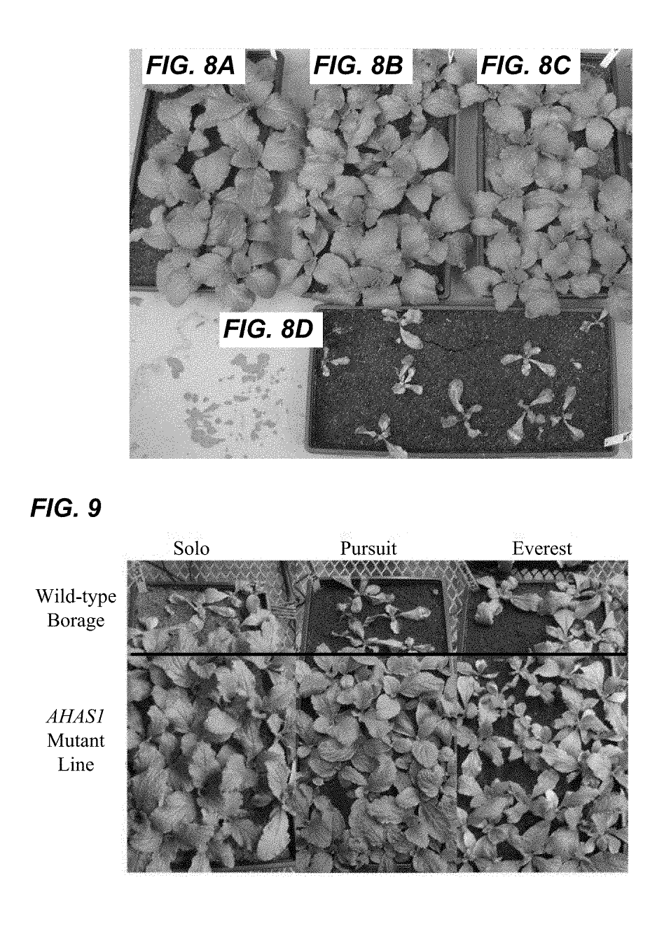

The herbicide dosage response test showed that 100% of the survival rate without any obvious injury was observed at four times the recommended “Solo” herbicide treatment (FIGS. 8A-D and FIG. 9). With the concentration increasing to eight times, the mutant plants showed injury symptoms. However, all of the wild type plants were completely wiped out by the herbicide at 2X concentrations (FIG. 9). This result indicated the mutant borage (AHAS1) was tolerant up to four times the recommended dosage of imidazolinone herbicides, whereas all wild type plants were not tolerant to the treatment.

Testing of different herbicides within Group 2 showed that besides imazamox, the AHAS1 mutant line was also highly tolerant to “Pursuit” (imazethapyr) with no obvious chemical damage (FIG. 10). Interestingly, as shown in FIG. 10, the AHAS1 mutant line also showed moderate tolerance to “Everest 2.0” herbicide (flucarbazone sodium). Other than that, the mutant line was sensitive to the other group 2 herbicides tested (Table 6).

TABLE 6
Responses of the AHAS1 mutant line towards
different group 2 herbicides

| Commercial Name | Active ingredient | Tolerance |
|---|---|---|
| Accent | nicosulfuron | No |
| Everest 2.0 | flucarbazone sodium | Yes |
| Express SG | tribenuron-methyl | No |
| Muster | ethametsulfuron methyl | No |
| Pinnacle SG | triflusulfuron methyl | No |
| PrePass XC | florasulam | No |
| Pursuit | imazethapyr | Yes |
| Solo | imazamox | Yes |

The herbicide dosage response test in this study provides direct evidence that the homozygous mutant line (AHAS1) is resistant to imidazolinones up to four times the agronomically recommended dosage. Based on visual observations, the treatment with 4X herbicide did not cause any obvious damage to the plant. In comparison with untreated control plants, the treated mutant plants showed similar growth and development (FIG. 20). However, with the concentration increasing to 8X, the mutant line showed sensitivity to the herbicide. A previous study (Duggleby and Pang, 2000, supra) showed that the mutation S653N of Arabidopsis AHAS could lead to 100 fold increase in tolerance to imidazolinones. Yet, the similar high level of resistance to the herbicide on the same mutation in borage was not observed in this study. The reason for the difference is not clear, but it may have something to do with the different genetic background of the two species. Arabidopsis possesses only one AHAS gene, while borage has two AHAS genes (AHAS1 and AHAS2) to support essential AHAS activity. If one of the two genes in borage is inhibited, the other gene might not be able to provide enough strength to tolerate a high level of the herbicide. However, the in vitro assays did show AHAS activity in the single gene mutant line could still maintain nearly 20% of the original activity at 625 μM of imazamox. Since the AHAS2 mutation was also discovered from another imidazolinone resistant line, it is possible to cross the homozygous AHAS1 mutant with the AHAS2 mutant plant to acquire offspring containing the two mutated AHAS genes. Then, the level of herbicide resistance would be expected to increase beyond the current level.

The result of herbicide type response test has showed that the M4 imidazolinone resistant borage (AHAS1 mutant) has an equally strong resistant level to both imazamox and imazethapyr, but zero tolerance to other herbicides except flucarbazone sodium (FIG. 9). This result was different from the studies of others that the S653 mutation only confers tolerance to imidazolinones, not any cross-tolerance to the other Group 2 herbicides. See e.g. Dietrich G E (1998) Imidazolinone resistant AHAS mutants, U.S. Pat. No. 5,767,361; Lee Y T, et al. Effect of mutagenesis at serine 653 of Arabidopsis thaliana acetohydroxyacid synthase on the sensitivity to imidazolinone and sulfonylurea herbicides. FEBS Letters 452 (1999) 341-345; Tan, S et al. Herbicidal inhibitors of amino acid biosynthesis and herbicide-tolerant crops. Amino Acids 30 (2006) 195-204).

In contrast to imazamox and imazethapyr treatments, the wild-type control treated by flucarbazone sodium showed less injury and damage (FIG. 9), indicating that the wild-type borage may naturally exhibit slight tolerance to flucarbazones by utilizing cytochrome P450 monooxygenases to convert the herbicide into non-toxic derivatives. See e.g. Yuan J S, et al. Non-target-site herbicide resistance: a family business. Trends in Plant Sciences 12 (2006) 12-13. However, the S651N mutation of the AHAS genes in borage enhances the level of tolerance to flucarbazones. This observation has never been reported before.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A method for controlling weeds in a vicinity of a borage plant, comprising applying a Group 2 herbicide to the weeds and the borage plant, wherein the borage plant encodes a mutated AHAS1 gene that includes a nucleic acid sequence according to SEQ ID NO: 14, wherein the mutated AHAS1 gene confers effective weed control of cultivated borage through use of the herbicide.

2. The method of claim 1, wherein the Group 2 herbicide is an imidazolinone herbicide selected from: imazethapyr, imazapic, imazamox, imazaquin, imazethabenz, imazapyr, a mixture of imazapyr and imazamox, and combinations thereof.

3. The method of claim 2, wherein the imidazolinone herbicide is selected from imazethapyr and imazamox and combinations thereof.

4. The method of claim 1, wherein the Group 2 herbicide is selected from flucarbazone sodium, imazethapyr and imazamox and combinations thereof.

5. The method of claim 1, wherein the borage plant encodes an AHAS1 enzyme according to SEQ ID NO: 19.

6. The method of claim 1, wherein the mutated AHAS1 gene encodes a polypeptide having an amino acid change S651N relative to a wild type borage AHAS1 polypeptide.

7. A borage plant comprises a mutated AHAS1 gene that encodes a polypeptide having an amino acid change S651N relative to a wild type borage AHAS1 polypeptide.

8. The borage plant of claim 7, wherein the borage plant encodes an AHAS1 enzyme according to SEQ ID NO: 19.

9. The borage plant of claim 7, wherein the plant was obtained by a process comprising mutating a borage plant with a chemical mutagen, selecting for Group 2 herbicide resistance, and determining homozygosis by KASP genotyping.

10. The borage plant of claim 9, wherein the herbicide is selected from one or more of flucarbazone sodium, imazethapyr and imazamox and combinations thereof.

11. The borage plant of claim 7, wherein the mutated AHAS1 gene has a nucleic acid sequence having greater than 94% homology with SEQ ID NO: 26 and that encodes a polypeptide having an amino acid change S651N relative to a wild type borage AHAS1 polypeptide.

12. A seed of the plant of claim 7.

13. A method for controlling weeds in a vicinity of a borage plant, comprising applying a Group 2 herbicide to the weeds and the borage plant, wherein the borage plant encodes a mutated AHAS2 gene that includes a nucleic acid sequence according to SEQ ID NO: 15, wherein the mutated AHAS2 gene confers effective weed control of cultivated borage through use of the herbicide.

14. The method of claim 13, wherein the Group 2 herbicide is an imidazolinone herbicide selected from: imazethapyr, imazapic, imazamox, imazaquin, imazethabenz, imazapyr, a mixture of imazapyr and imazamox, and combinations thereof.

15. The method of claim 14, wherein the imidazolinone herbicide is selected from imazethapyr and imazamox and combinations thereof.

16. The method of claim 13, wherein the Group 2 herbicide is selected from flucarbazone sodium, imazethapyr and imazamox and combinations thereof.

17. The method of claim 13, wherein the borage plant encodes an AHAS2 enzyme according to SEQ ID NO: 20.

18. The method of claim 13, wherein the mutated AHAS2 gene encodes a polypeptide having an amino acid change S647N relative to a wild type borage AHAS2 polypeptide.

19. A borage plant comprises a mutated AHAS2 gene that encodes a polypeptide having an amino acid change S647N relative to a wild type borage AHAS2 polypeptide.

20. The borage plant of claim 19, wherein the borage plant encodes an AHAS2 enzyme according to SEQ ID NO: 20.

21. The borage plant of claim 19, wherein the plant was obtained by a process comprising mutating a borage plant with a chemical mutagen, selecting for Group 2 herbicide resistance, and determining homozygosis by KASP genotyping.

22. The borage plant of claim 21, wherein the herbicide is selected from one or more of flucarbazone sodium, imazethapyr and imazamox and combinations thereof.

23. The borage plant of claim 13, wherein the mutated AHAS2 gene has a nucleic acid sequence having greater than 94% homology with SEQ ID NO: 27 and encodes a polypeptide having an amino acid change S647N relative to a wild type borage AHAS2 polypeptide.

24. A seed of the plant of claim 19.
